SEQUENCE LISTING (4 sequences)

SEQ ID NO: 1 - protein, 15 aa, Bos taurus
CGVPAIQPVLSGLSR

SEQ ID NO: 2 - protein, 8 aa, Electrophorus electricus
SELLVNTK

SEQ ID NO: 3 - protein, 5 aa, Sus scrofa
MAHYR

SEQ ID NO: 4 - protein, 5 aa, Gallus gallus
KVFGR

(19) United States
(12) Patent Application Publication
(10) Pub. No.: US 2020/0369716 A1
(43) Pub. Date: Nov. 26, 2020

(54) SOLID-PHASE SYNTHESIS OF PROTEIN-POLYMER CONJUGATES ON IMMOBILIZATION SUPPORTS

(71) Applicant: Carnegie Mellon University, Pittsburgh, PA (US)

(72) Inventors: Hironobu Murata, Pittsburgh, PA (US); Alan J. Russell, Gibsonia, PA (US)

(21) Appl. No.: 16/635,343

(22) PCT Filed: Aug. 1, 2018
(86) PCT No.: PCT/US18/44859

§ 371 Date: Jan. 30, 2020

Related U.S. Application Data
(60) Provisional application No. 62/605,104, filed on Aug. 1, 2017.

(51) Int. Cl. C07K 1/04; C07K 1/107; C12N 11/087; C08B 37/00

(52) U.S. Cl. CPC C07K 1/04; C07K 1/1077; C08B 37/0039; C12Y 301/01007; C12Y 304/21001; C12N 11/087

(57) ABSTRACT

Materials and methods for generating protein-polymer conjugates on solid supports are provided herein. The methods can include, for example, reversibly immobilizing a protein on a solid support, modifying the protein by adding polymer subunits, and releasing the protein-polymer conjugate from the solid support.

### CROSS-REFERENCE TO RELATED APPLICATIONS

This application claims benefit of priority from U.S. Provisional Application Ser. No. 62/605,104, filed on Aug. 1, 2017, the disclosure of which is incorporated herein by reference in its entirety.

### TECHNICAL FIELD

This document provides materials and methods for rapidly and consistently generating protein-polymer conjugates on solid supports, including materials and methods that involve reversibly immobilizing a protein on a solid support, modifying the protein by adding polymer subunits, and releasing the protein-polymer conjugate from the solid support.

### BACKGROUND

Solid-phase synthesis is a method in which substances are immobilized on a solid support and then reacted in a reactant solution. After washing away excess reactant and side products from the beads, the modified substance can be released from the support. Solid-phase synthesis has been widely utilized for the synthesis of peptides and nucleic acids, for example.

### SUMMARY

The design of customized protein-polymer conjugates has the potential to open new vistas of research and applications in the pharmaceutical, food, bioenergy, and biotechnology industries. Current methods for solid-phase synthesis of biomacromolecules are performed in organic solvents, however. The use of solid-phase synthesis methods to chemically modify an immobilized protein would require the development of synthetic techniques that operate in aqueous media, in order to minimize denaturation and deactivation of the protein.

Further, since protein-polymer conjugates typically are synthesized in monophasic solutions, significant skill and experience in both organic synthesis and purification is required for preparation of such conjugates. A device to automate the growth of polymers from protein surfaces would be dependent on the development of reversible immobilization supports through which a protein could be reversibly displayed for automated modification in a batch reactor or continuous flow device.

This document is based, at least in part, on the successful development of a reversible protein immobilization solid support that can be used to display proteins for subsequent modification, grown-from polymerization, purification, and detachment in aqueous media. Thus, this document provides a solid phase that can be attached via any reversible bond to any site on a protein that allows the protein to remain active when released from the solid phase. This document also provides methods for generating such modified proteins. Non-limiting examples of applicable chemistries are described herein. In some embodiments, for example, the methods provided by this document employ reversible immobilization techniques that utilize disulfide bonds, dialkyl maleic anhydride (DMA), or ligand affinity for binding a protein to a solid support. In some cases, polystyrene, agarose, cross-linked polyethylene oxide, magnetic beads, and nickel-imidodiacetic acid resins can be used as solid supports.

The materials and methods described herein have been tested experimentally on protein-polymer conjugation, and the results are discussed below. These novel methods can shorten the entire processing time required for synthesis of polymer-protein conjugates from days to hours. In addition, the methods can be applied to automated and combinatorial synthesis of protein-polymer conjugates, as well as other synthetic applications.

In a first aspect, this document features a method for generating a protein-polymer conjugate. The method can include attaching a protein of interest to a solid support adapted for coupling to the protein of interest, contacting the protein of interest with an initiator (e.g., an initiator that modifies a functional group on the protein of interest), and coupling a first monomer subunit of a polymer to the protein of interest via the initiator and, optionally, coupling further monomer subunits to the protein via the first monomer subunit, thus generating the protein-polymer conjugate. The solid support can be modified for reversible coupling to the protein of interest, such that the method includes reversibly coupling the protein of interest to the solid support. In such cases, the method can further include releasing the protein-polymer conjugate from the solid support. In some embodiments, the method can include non-reversibly attaching the protein of interest to the solid support.

The protein of interest can be an enzyme, a hormone, a cytokine, an antibody, an antigen, an or anti-coagulation protein. For example, the protein of interest can be an enzyme selected from the group consisting of a protease, an esterase, a dehydrogenase, a hydrolase, and a kinase.

The solid support can be hydrophilic. The solid support can include a polystyrene resin, agarose beads, cross-linked polyethylene glycol (PEG) beads, an affinity resin, or magnetic (nano)-beads. In some cases, the solid support can include agarose beads.

The solid support can have a functional group that includes a maleic anhydride, and the method can include attaching the protein of interest to the solid support via reaction of the maleic anhydride with an amino group on the protein of interest. The maleic anhydride can be dimethyl maleic anhydride (DMA). The method can include attaching the protein of interest to the solid support at a pH between 5.5 and 6.5, such that the protein of interest is attached to the solid support via a covalent bond formed between the maleic anhydride and the N-terminal amino group of the protein. The method can further include exposing the protein-polymer conjugate on the solid support to a pH between about 3 and about 4, such that the protein-polymer conjugate is released from the solid support. The method can include, prior to exposing the protein-polymer conjugate on the solid support to a pH between about 3 and about 4, contacting the protein-polymer conjugate on the solid support with agarase.

The solid support can have a functional group that includes a disulfide bond, and the method can include attaching the protein of interest to the solid support via reaction of the disulfide bond with a thiol group of the protein of interest. The method can further include contacting the protein-polymer conjugate on the solid support with a reducing agent (e.g., dithiothreitol), such that the protein-polymer conjugate is released from the solid support.

The solid support can have a ligand with affinity for the protein of interest, and the method can include attaching the protein of interest to the solid support via interaction with the ligand. The ligand can be a substrate or an inhibitor of the protein of interest.

The solid support can include a nickel-imidodiacetic acid (Ni-IDA) resin, and the method an include attaching the protein of interest to the solid support via interaction of the Ni-IDA resin with a histidine residue of the protein. The method can further include exposing the protein-polymer conjugate on the solid support to a pH of about 2.5, such that the protein-polymer conjugate is released from the solid support. The method can further include contacting the protein-polymer conjugate on the solid support with imidazole or ethylenediamenetetraacetic acid (EDTA), such that the protein-polymer conjugate is released from the solid support.

The initiator can be an atom transfer radical polymerization (ATRP) initiator [e.g., N-2-bromo-2-methylpropanoyl-β-alanine N′-oxysuccinimide bromide (NHS—Br)].

The protein of interest can be an enzyme, and the method can further include, prior to or simultaneously with contacting the protein of interest with the initiator, the polymer subunit, or both, contacting the protein of interest with a substrate for the enzyme. The polymer can be a zwitterionic polymer. The polymer can include a methacrylate, acrylate, acrylamide, styrenic, or acrylamide-styrenic or a combination thereof. The polymer can be poly(carboxybetaine methacrylate) (pCBMA). The method can further include at least one washing step selected from the group consisting of: washing uncoupled protein of interest away from the solid support prior to adding the initiator, washing excess initiator away from the protein of interest on the solid support prior to coupling the first polymer subunit to the protein of interest, and washing uncoupled polymer subunits away from the protein-polymer conjugate on the solid support. The attaching, contacting, and coupling steps can be conducted in an automated system (e.g., a flow through system).

In another aspect, this document features a method for generating a protein-polymer conjugate, where the method includes (a) coupling a protein of interest to a solid support, where the solid support includes DMA-modified agarose beads, and where the coupling results in covalent attachment of the protein to the support via a bond between the DMA and an amino group on the protein, (b) contacting the protein of interest on the solid support with an ATRP initiator, where the ATRP initiator is NHS—Br, and where the NHS—Br modifies amino groups on the protein of interest, (c) coupling repeating subunits of a polymer to the protein of interest via the ATRP initiator, thus generating the protein-polymer conjugate, and (d) releasing the protein-polymer conjugate from the solid support by exposing the protein-polymer conjugate on the solid support to a pH between about 3 and about 4, such that the protein-polymer conjugate is released from the solid support. The protein of interest can be an enzyme, a hormone, a cytokine, an antibody, an antigen, an or anti-coagulation protein. In some cases, the protein of interest can be an enzyme selected from the group consisting of a protease, an esterase, a dehydrogenase, a hydrolase, and a kinase. The method can include coupling the protein of interest to the solid support at a pH between 5.5 and 6.5, such that the protein of interest is attached to the solid support via a covalent bond formed between the DMA and the N-terminal amino group of the protein. The method can further include, prior to exposing the protein-polymer conjugate on the solid support to a pH between about 3 and about 4, contacting the protein-polymer conjugate on the solid support with agarase. The protein of interest can be an enzyme, and the method can further include, prior to or simultaneously with contacting the protein of interest with the initiator, the repeating subunits of the polymer, or both, contacting the protein of interest with a substrate for the enzyme. The polymer can be a zwitterionic polymer. The polymer can include a methacrylate, acrylate, acrylamide, styrenic, or acrylamide-styrenic or a combinations thereof. The polymer can be pCBMA. The method can further include washing excess initiator away from the protein of interest on the solid support, prior to coupling the repeating subunits of the polymer to the protein of interest. One or more steps of the method can be conducted in an automated system (e.g., a flow through system). The method further can include washing uncoupled protein of interest away from the solid support prior to adding the initiator, washing excess initiator away from the protein of interest on the solid support prior to coupling the repeating polymer subunits to the protein of interest, and washing uncoupled polymer subunits away from the protein-polymer conjugate on the solid support prior to the releasing step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a diagram showing a scheme for synthesizing dialkyl maleic anhydride-modified agarose beads.

FIG. 2 is a diagram showing the steps in solid-phase synthesis of protein-polymer conjugates by Protein-Atom transfer radical polymerization on Reversible Immobilization Supports (PARIS). Protein is first immobilized onto DMA-agarose beads at pH 6-8 through surface accessible primary amines. Remaining primary amines on the protein are then modified with an ATRP initiator (e.g., NHS—Br), followed by surface-initiated ATRP, to create functional protein-polymer conjugates. The conjugates are then detached from the DMA-agarose beads below pH 6, and can be passed through a filter in pure form.

FIGS. 3A-3E are a series of FT-IR spectra for dried beads at each step of the PARIS synthesis. FIG. 3A: DMA bead (1807 and 1764 cm⁻¹ (ν_{C═O}: anhydride) and 1647 cm⁻¹ (υ_{C=N}: imine and υ_{C=O}: ester). FIG. 3B: CT immobilized DMA bead (1645 cm⁻¹ (ν_{C=O}: amide I), 1546 cm⁻¹ (δ_{N—H}: amide II)). FIG. 3C: CT-ATRP initiator on DMA bead. (1645 cm⁻¹ (ν_{C=O}: amide I), 1546 cm⁻¹ (δ_{N—H}: amide II)). FIG. 3D: CT-poly(carboxybetaine methacrylate) (pCBMA) conjugate on DMA bead 1727 cm⁻¹ ν_{C=O}: ester), 1643 cm⁻¹ (ν_{C=O}: amide I), 1590 (νₐₛ:—COO—), 1550 cm⁻¹ (δ_{N—H}: amide II)). FIG. 3E: DMA bead after CT-pCBMA cleavage (1764 cm⁻¹ (ν_{C=O}: anhydride), 1727 cm⁻¹ (ν_{C=O}: ester), 1643 cm⁻¹ (ν_{C=O}: amide I), 1590 (νₐₛ:—COO⁻), 1550 cm⁻¹ (δ_{N—H}: amide II)).

FIGS. 4A-4G show the pH dependence of binding and releasing affinities of GGCy3 and Cy5.5 amine to DMA-agarose beads. FIG. 4A shows the structure of Glycyl-glycyl-Cy3 as a model for the N-terminus of a polypeptide. FIG. 4B shows the structure of Cy5.5 amine as a model for a Lys side chain. FIG. 4C shows a scheme for a binding and releasing reaction between amino dyes and DMA beads. FIG. 4D is a graph plotting the pH dependence of the binding rate of GGCy3 to DMA beads (pH 5, squares; pH 6, circles; pH 7, diamonds; pH 8, triangles). FIG. 4E is a graph plotting the pH dependence of the binding rate of Cy5.5 amine to DMA beads (pH 5, squares; pH 6, circles; pH 7, diamonds; pH 8, triangles). FIG. 4F is a graph plotting the pH dependence of the releasing rate of GGCy3 from DMA beads (pH 3, triangles; pH 4, diamonds; pH 5, squares; pH 6, circles). FIG. 4G is a graph plotting the pH dependence of the releasing rate of Cy5.5 amine from DMA beads (pH 3, triangles; pH 4, diamonds; pH 5, squares; pH 6, circles). Error bars represent standard deviations from triplicate measurements.

FIG. 5 is a diagram of a method for synthesis of the N-terminus model dye, Glycyl-glycyl-Cy3. Cy3 amine, HRMS (m/z): [M−H]⁺ calcd. for C₃₆H₅₂N₄O₅²⁺, 556.41; found, 556.84.

FIG. 6 shows a ¹H NMR (300 MHz, CDCl₃) spectrum of Boc-Gly-Gly-NHS. Boc-Gly-Gly-NHS, mp: 139-142° C., ¹H NMR (300 MHz, DMSO-d₆) δ 1.47 (s, 9H, Boc-), 2.87 (s, 4H, Succinimide), 3.88 (d, 2H, J=6.0 Hz, Gly^{α}), 4.45 (d, 2H, J=6.0 Hz, Gly″), 5.21 (broad s, 1H, NH) and 6.83 (broad s, 1H, NH) ppm; ¹³C NMR (75 MHz, DMSO-d₆) δ 24.8, 25.0, 27.0, 27.6, 27.8, 28.4, 37.7, 42.5, 77.7, 155.3, 165.8, 169.5, 169.8, 172.3 ppm; IR (KBr pellet) 2980, 2938, 1821, 1789, 1741, 1702, 1665, 1575 and 1514 cm⁻¹.

FIG. 7 shows a ¹H NMR (300 MHz, CDCl₃) spectrum of Boc-GG-Cy3. Boc-Gly-Gly-Cy3, oily compound, ¹H NMR (300 MHz, CDCl₃) δ 1.41 (m, 9H, Boc- and 4H, 2×CH₂), 1.58 (m, 6H, 3×CH₂), 1.73 (s, 12H, Cy3-CH₃), 1.83 (m, 2H, CH₂), 2.42 (t, J=7.2 Hz, C═OCH2), 3.27 (m, 4H, 2×NHCH₂), 3.74 (s, 3H, Cy3 N—CH₃), 3.92 (d, 2H, J=5.7 Hz, Glyn), 3.99 (d, 2H, J=5.7 Hz, Gly^{α′}), 6.25 (broad s, 1H, Boc-NH), 6.82 (d, 1H, J=13.2 Hz, C═CH), 6.92 (d, 1H, J=13.2 Hz, C═CH), 7.16-7.45 (m, 8H, Cy3-Ar H), 7.90 (m, 2H, amide), 8.15 (broad t, 1H, J=4.2 Hz, amide), 8.41 (t, 1H, J=13.2 Hz, CH═CH—CH) ppm; ¹³C NMR (75 MHz, DMSO-do) S 25.0, 25.5, 26.0, 26.7, 26.8, 27.7, 27.8, 28.2, 28.5, 28.8, 35.6, 35.7, 38.5, 42.9, 44.3, 48.8, 48.9, 79.3, 103.2, 111.1, 121.5, 122.5 125.2, 125.6, 128.0, 128.9, 140.0, 141.5, 142.2, 150.2, 156.0, 169.2, 170.4, 173.4, 173.8 ppm; IR (NaCl plate) 2950, 2925, 2856, 1655, 1559, 1493, 1458, 1415 and 1371 cm⁻¹; HRMS (m/z): [M−H]⁺ calcd. for C₄₅H₆₅N₆O₅, 769.501; found, 770.051.

FIG. 8 shows a ¹H NMR (300 Mhz, CDCl₃) spectrum of GGCy3. Gly-Gly-Cy3, oily compound, 1H NMR (300 MHz, CDCl₃) δ 1.4-1.9 (broad, 14H, 7×CH₂ and 12H, Cy3-CH₃), 2.5 (broad, 2H, CH₂), 3.2 (broad, 4H, 2×CH₂), 3.8 (broad, 3H, Cy3 N—CH₃), 4.0-4.3 (broad, 2H, CH₂ and 4H, 2×Glya), 6.8 (broad, 2H, CH═CH), 7.3 and 7.4 (broad, 8H Cy3-Ar H), 8.2 (broad, 1H, amide), 8.4 (broad, 1H, CH═CH—CH), 8.6 (broad, 2H, amide) ppm; ¹³C NMR (75 MHz, DMSO-d₆) δ 25.2, 25.7, 26.1, 26.8, 28.0, 28.2, 28.7, 28.9, 29.0, 29.1, 29.7, 34.0, 35.0, 36.1, 38.7, 39.7, 42.9, 49.0, 49.2, 103.9, 105.6, 116.0, 125.7, 127.5, 128:5, 131.2, 140.5, 141.9, 169.3, 170.1, 173.8 ppm; IR (NaCl plate) 2956, 2924, 2853, 1712, 1651, 1557, 1493, 1456, 1416 and 1376 cm⁻¹; FIRMS (m/z): [M−2H]⁺ calcd. for C₄₀H₅₇N₆O₃²⁺, 670.46; found, 670.94.

FIG. 9A shows a schematic of a method for reversible protein immobilization on DMA-agarose beads and ATRP initiator modification. FIGS. 9B and 9C are graphs plotting chymotrypsin release kinetics as a function of pH (3-6) after initial immobilization at pH 6.0 (FIG. 9B, determined by microBCA protein assay) or pH 8.0 (FIG. 9C, determined by microBCA assay). In both plots, release was performed by incubation at pH 3 (triangles), pH 4 (diamonds), pH 5 (squares), and pH 6 (circles). The amount of recoverable protein and release rate increased as pH decreased. Error bars represent standard deviation from triplicate measurements.

FIG. 10 is a graph depicting the stability of native chymotrypsin (CT) in 20 mM citrate buffer (pH 3) at 25° C. Chymotrypsin's residual activity was measured at pH 8 (100 mM sodium phosphate buffer) using suc-AAPF-pNA as a substrate at specified time points after incubation in pH 3 releasing buffer. Chymotrypsin maintained full activity even after 3 hours, indicating that the lower pH of releasing buffer did not cause enzymatic activity loss. Error bars represent standard deviation from triplicate measurements.

FIGS. 11A-11C are a series of MALDI-ToF mass spectroscopy plots of native CT and initiator modified CT (CT-Br), initially immobilized at pH 6.0 and pH 8.0, to determine the number of modifications. FIG. 11A is a spectrum for native CT. CTBr was modified with 13 initiators at pH 6.0 (FIG. 11B), and 11 initiators at pH 8.0 (FIG. 11C).

FIGS. 12A-12C are a series of MALDI-ToF mass spectroscopy plots showing the determination of N-terminus selectivity. The presence of an N-terminus peptide fragment at 1554.63 m/z was observed in the spectra for native CT (FIG. 12A) and CT-initiator immobilized at pH 6.0 (FIG. 12B), but the same peptide fragment was not observed in the spectrum for CT-initiator immobilized at pH 8.0 (FIG. 12C), indicating N-terminal selective binding to the DMA beads.

FIG. 13 is a ¹H NMR spectrum of released CT-pCBMA by PARIS in D₂O to verify the polymer chemical structure.

FIG. 14 is a FT-IR spectrum of released CT-pCBMA by PARIS. The spectrum was measured using a KBr pellet.

FIG. 15A is a schematic showing steps in a PARIS method for producing a pCBMA-protein conjugate. FIG. 15B is a graph plotting the activity of released PARIS-synthesized CT-pCBMA conjugates, solution synthesized CT-pCBMA conjugates, and native CT over time, based on thermostability during incubation at 50° C. Open circles, CT-pCBMA immobilized at pH 6.0 and polymerized for 2 hours; open diamonds, CT-pCBMA immobilized at pH 8.0 and polymerized for 2 hours; open triangles, CT-pCBMA by solution synthesis; solid circles, native CT. Samples were incubated in 100 mM sodium phosphate buffer (pH 8.0, 50° C.) at 3.9 μM CT, and residual activity was measured over 6 hours. All CT-pCBMA conjugates showed enhanced thermostability compared to native CT. CT-pCBMA immobilized at pH 6.0 with a free N-terminus showed the highest thermostability. Error bars represent standard deviation from triplicate measurements.

FIGS. 16A-16D are a series of histograms plotting the particle size distribution of CT-pCBMA conjugates by number. FIG. 16A, fixed monomer concentration of 25 mM by solution method (polymerization time for 5 minutes, dots; 10 minutes, dashed dots; 20 minutes, dashes; 40 minutes, long dashes; 60 minutes, solid line; native CT, solid line to the left). FIG. 16B, fixed monomer concentration of 25 mM by PARIS (5 minutes, dots; 10 minutes, dashed dots; 20 minutes, dashes; 40 minutes, long dashes; 60 minutes, solid line; native CT, solid line to the left). FIG. 16C, increasing monomer concentrations for a 60 minute reaction time by solution method (12.5 mM, dashes; 25 mM, solid line; 50 mM, dots; 100 mM, long dashes). FIG. 16D, increasing monomer concentrations for a 60 minute reaction time by PARIS (12.5 mM, dashes; 25 mM, solid line; 50 mM, dots; 100 mM, long dashes).

FIGS. 17A-17D are a series of gel permeation chromatography (GPC) traces of cleaved pCBMA from CT-pCBMA conjugates. FIG. 17A, fixed monomer concentration of 25 mM by solution method. FIG. 17B, fixed monomer concentration of 25 mM by PARIS. For FIGS. 17A and 17B, the reaction times were 5 minutes, 10 minutes, 20 minutes, 40 minutes, and 60 minutes, as indicated. FIG. 17C, increasing monomer concentrations for a 60 minute reaction time by solution method. FIG. 17D, increasing monomer concentrations for a 60 minute reaction time by PARIS. For FIGS. 17C and 17D, the monomer concentration were 12 mM, 25 mM, 50 mM, and 100 mM, as indicated.

FIGS. 18A-18C are a series of spectra showing the presence of an N-terminus peptide fragment at 585.20 m/z in the spectra for native Lysozyme (FIG. 18A), Lysozyme-initiator immobilized at pH 6.0 (FIG. 18B), and Lysozyme-initiator immobilized at pH 8.0 (FIG. 18C), suggesting that the N-terminus did not react with either the DMA beads or the bromine-initiator. Exposed surface accessibility calculations showed that the α-amino group was buried and was most likely unable to react.

FIGS. 19A-19C are a series of spectra showing the presence of an N-terminus peptide fragment at 555.99 m/z in the spectra for native avidin (FIG. 19A), avidin-initiator immobilized at pH 6.0 (FIG. 19B), and avidin-initiator immobilized at pH 8.0 (FIG. 19C), suggesting that the N-terminus did not react with either the DMA beads or the bromine-initiator. Exposed surface accessibility calculations showed that the α-amino group was buried and was most likely unable to react.

FIGS. 20A-20C are a series of spectra showing the presence of N-terminus peptide fragment at 904.97 m/z in the spectra for native AChE (FIG. 20A) and AChE-initiator immobilized at pH 6.0 (FIG. 20B), and showing the lack of the same peptide fragment in the spectrum for AChE-initiator immobilized at pH 8.0 (FIG. 20C), indicating N-terminal selective binding to the DMA beads.

FIGS. 21A-21C are a series of spectra showing the presence of an N-terminus peptide fragment at 676.97 m/z in the spectra for native Uox (FIG. 21A) and Uox-initiator immobilized at pH 6.0 (FIG. 21B), and showing the lack of the same peptide fragment in the spectrum for Uox-initiator immobilized at pH 8.0 (FIG. 21C), indicating N-terminal selective binding.

FIGS. 22A-22C are a series of MALDI-ToF mass spectroscopy plots for native lysozyme and initiator modified lysozyme (Lyz-Br) initially immobilized at pH 6.0 and pH 8.0, to determine the number of modifications. FIG. 22A, native lysozyme; FIG. 22B, lysozyme-initiator immobilized at pH 6.0 (6 initiators); and FIG. 22C, lysozyme-initiator immobilized at pH 8.0 (6 initiators).

FIGS. 23A-23C are a series of MALDI-ToF mass spectroscopy plots for native avidin and initiator modified avidin (Av-Br) initially immobilized at pH 6.0 and pH 8.0, to determine the number of modifications. FIG. 23A, native avidin; FIG. 23B, avidin-initiator immobilized at pH 6.0 (8 initiators); and FIG. 23C, avidin-initiator immobilized at pH 8.0 (8 initiators).

FIGS. 24A-24C are a series of MALDI-ToF mass spectroscopy plots for native acetylcholinesterase and initiator modified acetylcholinesterase (AChE-Br), initially immobilized at pH 6.0 and pH 8.0, to determine the number of modifications. FIG. 24A, native AChE [M+3H]³⁺; FIG. 24B, AChE-initiator immobilized at pH 6.0 (11 initiators), and FIG. 24C, AChE-initiator immobilized at pH 8.0 (9 initiators).

FIG. 25 is a graph plotting the impact of agarase pre-incubation on CT-PCBA release from DMA beads. Pre-incubation with agarase, circles; without agarase pre-incubation, triangles. Error bars represent standard deviation from triplicate measurements.

FIGS. 26A and 26B are a pair of graphs plotting the irreversible inactivation of AChE at decreasing pH. FIG. 26A, incubation in 20 mM citrate buffer (pH 3-6) at room temperature. FIG. 26B, incubation in 100 mM sodium phosphate buffer (pH 5 and pH 6) at room temperature. Error bars represent standard deviation from triplicate measurements.

FIG. 27A shows a set up for a flow reactor for automated one-pot rapid PARIS protein-polymer conjugate synthesis and properties. FIG. 27B is a graph plotting irreversible inactivation of native CT and CT-pCBMA at 50° C. CT-pCBMA synthesized in the flow reactor immobilized at pH 6.0 (open circles) or pH 8.0 (open diamonds) and native CT (filled circles) were incubated in 100 mM sodium phosphate buffer (pH 8.0, 50° C.) at 3.9 μM CT for 6 hours. Both CT-pCBMA conjugates showed enhanced thermostability as compared to native CT. CT-pCBMA immobilized at pH 6.0 via the N-terminus showed the highest thermostability, similar to batch mode studies. Error bars represent standard deviation from triplicate measurements.

FIGS. 28A, 28B, and 28D-28I are a series of diagrams illustrating the steps in a representative automated flow through PARIS method for making an exemplary protein-polymer conjugate (CT-CBMA). FIG. 28A shows an initial set up with solid support-containing column 100 adapted to receive a protein (in this case, CT 120), initiator 130, ligand 140, polymer (in this case, CBMA 150), washing buffer 160, substrate 170, and releasing buffer 170, under the control of processor 180. Output from the solid support can be monitored by UV-vis detector 190 and size monitoring instrument 195 (which, in this case, utilizes multi-angle light scattering). As depicted in FIG. 28B, CT protein was applied to solid support-containing column 100, and CT immobilization on the solid support was determined based absorbance at 280 nm. FIG. 28C is a graph plotting absorbance at 280 nm for CT applied to agarose beads or DMA-modified agarose beads, as indicated. FIG. 28D, the column was washed with washing buffer 160 and substrate 170, which can prevent the CT active site from becoming modified by initiator in the next step. CT release and activity were monitored at 280 nm and 410 nm, respectively. FIG. 28E, initiator 130 was added to the immobilized protein on the solid support, together with substrate 170. FIG. 28F, washing buffer 160 and substrate 170 were passed over the column to remove excess initiator. FIG. 28G, ligand 140 and polymer 150 (CBMA), as well as substrate 170, were added to the column; excess ligand and polymer were subsequently washed away with washing buffer 160 (and substrate 170) as shown in FIG. 28H. Finally, as illustrated in FIG. 28I, the protein polymer conjugate was removed from solid support-containing column 100 by releasing buffer 110, and the size and molecular weight of the conjugate were assessed (e.g., using instrument 195).

### DETAILED DESCRIPTION

Facile automated biomacromolecule synthesis is at the heart of the ability to blend the synthetic and biologic worlds. Protein-polymer conjugates have been synthesized in solution in multi-step, multi-day processes that couple innovative chemistry with challenging purification strategies. This document provides materials and methods for the first generation of protein-polymer hybrids synthesized by Protein-ATRP on Reversible Immobilization Supports (PARIS). As described in the Examples below, modified agarose beads were used to covalently and reversibly immobilize proteins via amine-specific interactions. The reversibly immobilized proteins were then modified with a protein-reactive ATRP initiator, and after ATRP, protein-polymers were released and analyzed. The process was automated, and synthesis and purification were completed in just six hours. Comparison of PARIS with liquid-phase methods demonstrated that PARIS is an effective, rapid, and simple method to revolutionize and automate the generation of protein-polymer conjugates.

Through work with PEGylated proteins (Abuchowski et al., J. Bio. Chem. 252:3582-3586, 1976; and Abuchowski et al., J. Control. Rel. 252:3578-3581, 1977), the merger of the synthetic and biologic worlds has saved countless lives and driven the application of biocatalysis in a variety of industries (Wilson, Macromol. Chem. Phys. 218:1600595, 2017; O'Sullivan et al., Value Heal. 11: A437, 2008; Nesbitt et al., Inflamm. Bowel Dis. 13:1323-1332, 2007; Hills, Eur. J. Lipid Sci. Technol. 105:601-607, 2003; and Griengl et al., Trends Biotechnol. 18:252-256, 2000). Protein PEGylation takes place in solution-based “grafting-to” syntheses (Harris and Chess, Nat. Rev. Drug Discov. 2:214-221, 2003; and Isarov et al., Biomacromolecules 17:641-648, 2016) that usually require a large excess of polymer and are not easily controlled, and in which the density of modification is subject to steric hindrance (Yang et al., Enzyme Microb. Technol. 18:82-89, 1996).

More recently, alternative routes to engineer the structure and function of proteins by growing polymers from their surfaces have been developed (Cobo et al., Nat. Mater. 14:143-159, 2014; Gong et al., Bioconjug. Chem. 26:1179-1181, 2015; Lele et al., Biomacromolecules 6:3380-3387. 2005; Cummings et al., Biomaterials 34:7437-7443, 2013; and Murata et al., Biomacromolecules 14:1919-1926, 2013). A “grafting-from” approach uses atom-transfer radical polymerization (ATRP) from initiators that have been covalently attached to a protein. The high polymer grafting density and the potential for site-specific polymer growth achieved by protein-ATRP enabled the synthesis of rationally designed functional protein-polymer conjugates (Heredia et al., J. Am. Chem. Soc. 127:16955-16960, 2005), with dramatically enhanced stability (Cummings 2013, supra; Cummings et al., Biomacromolecules 18(2):576-586, 2017); and Cummings, Biomacromolecules 15(3):763-771, 2014) and therapeutic potential (Zhao et al., Polymer 66:A1-A10, 2015; and Schulz et al., Adv. Mater. 28:1455-1460, 2016). Growing polymers via ATRP from soluble surface-initiated proteins is effective, but the need to remove unreacted initiators, monomers, and catalysts in multiple purification steps limited automation of the process, and also limited its availability. It can take many days of careful synthesis and purification to generate just one protein-polymer variant. As described herein, this challenge has now been overcome by growing polymers from proteins that have been reversibly immobilized onto a solid surface.

The methods described herein can be used to reversibly immobilize entire proteins on a solid support, and then subsequently react the immobilized proteins with ATRP initiators (or other compounds of interest) before site-specific polymer growth. The resulting protein-polymer conjugates can then be released in a pure form from the solid support. These methods, termed “PARIS” as noted above, can powerfully transform the synthesis and impact of protein-polymer conjugates.

A variety of chemistries can be used to reversibly bind proteins to solid supports. These include, for example, non-covalent interactions (Bahulekar et al., Enzyme Microb. Technol. 13:858-868, 1991) and hydrophobic adsorption (Caldwell et al., Biotechnol. Bioeng. 18:1573-1588, 1976), although such protein-support interactions generally are weak and protein-specific. Immobilized metal-affinity chromatography can be used for purification of recombinant proteins containing an affinity tag, such as polyhistidine (Porath, Protein Expr. Purif. 3:263-281, 1992). In addition, stable covalent disulfide bonds between free thiol groups on proteins and solid supports can be reduced to release bound proteins (Laurell et al., Anal. Biochem. 81:336-345, 1977).

The PARIS synthesis methods provided herein are broadly applicable for synthesis of protein-polymer conjugates, and employ a covalent and reversible coupling chemistry that can be used with almost any protein. In general, a solid support can be functionalized with DMA, which then can react with primary amino groups on essentially any protein. This reaction is reversible and pH dependent, with the complex dissociating at low pH (Nieto and Palacián, Biochim. Biophys. Acta (BBA)/Protein Struct. Mol. 749:204-210, 1983). The non-limiting examples below describe the targeted immobilization of a protein's N-terminal α-amino group to DMA-modified agarose beads, followed by ATRP from subsequently initiator-modified ε-amino groups on the protein surface, prior to bioconjugate release by reducing the pH. These PARIS-based methods for synthesizing protein-polymer conjugates opens the door to automated combinatorial syntheses and high throughput screening of next generation protein-polymer hybrids.

Thus, this document provides methods for generating polypeptide-polymer conjugates, as well as the solid supports, complexes, and conjugates used in and generated by the methods provided herein.

Any suitable solid support to which a polypeptide can be bound can be used in the methods provided herein. In some cases, solid supports can be modified by adding a functional group to which a protein can subsequently be bound. Examples of such functional groups include, without limitation, functional groups containing disulfide bonds and functional groups containing a maleic anhydride (e.g., a dialkyl maleic anhydride). The solid supports that can be used in the methods described herein include, without limitation, magnetic beads or polymer beads and resins made from materials such as polystyrene or agarose. In some cases, planar glass surfaces also can be used. Since the amount of space available on a solid support for protein coupling is determined by the surface area, porous, high surface area materials such as polystyrene or crosslinked agarose (e.g., SEPHAROSE®) beads can, in some embodiments, be particularly useful.

In some cases, the solid support can be commercially obtained in a modified form. Alternatively, an unmodified solid support can be obtained and then modified as described herein (e.g., by addition of DMA groups) before coupling to a protein. In some cases, an affinity resin bearing a ligand for the polypeptide to be modified by polymer conjugation can be used.

Any polypeptide can be coupled to a solid support for generating a protein-polymer conjugate as provided herein. Suitable polypeptides, include, without limitation, therapeutic proteins and proteins used in industrial applications. In some cases, the polypeptide can be a recombinant polypeptide. Therapeutic proteins for the treatment of a variety of diseases are known in the art (see, e.g., Dimitrov, Methods Mol. Biol. 899:1-26, 2012, which is incorporated herein by reference), and can be conjugated to a polymer to form a protein-polymer conjugate as described herein. In some embodiments, for example, the polypeptide can be an antibody (e.g., a monoclonal antibody or a fragment thereof), a Fc fusion protein, an enzyme, an anti-coagulation protein, a blood factor, a bone morphogenetic protein, a growth factor, an interferon, an interleukin, a thrombolytic agent, a protein or peptide antigen, or a hormone.

In some embodiments, the protein can be an enzyme. For example, the protein can be a protease or peptidase such as chymotrypsin, trypsin, pepsin, thrombin, plasmin, or elastase; an esterase such as a cholinesterase, thioesterase, phosphatase, or nuclease; a dehydrogenase such as acetaldehyde dehydrogenase, alcohol dehydrogenase, lactate dehydrogenase, or isocitrate dehydrogenase; a hydrolase such as a lipase, phosphatase, or glycosidase; or a kinase such as a phosphatidylinositol kinase, a mitogen-activated protein kinase, or a cyclin dependent kinase.

In some cases, the protein can be an antibody or a fragment thereof, or a fusion protein containing an Fc antibody fragment and a second polypeptide (e.g., an interleukin or interleukin receptor, an immune checkpoint molecule such as CTLA-4 or PD-1, a cytokine or cytokine receptor such as tumor necrosis factor receptor (TNFR) or granulocyte macrophage colony stimulating factor (GM-CSF), a growth factor such as epidermal growth factor (EGF) or fibroblast growth factor (FGF), or a hormone such as glucagon, insulin, or GLP-1, and analogs and variants thereof).

In some embodiments, the protein can be an anti-coagulation protein (e.g., tissue plasminogen activator or heparin), a blood factor (e.g., Factor II, Factor V, Factor VII, Factor VIII, Factor IX, Factor X, Factor XI, Factor XIII, protein C, protein S, von Willebrand Factor, or antithrombin III), a bone morphogenetic protein (BMP; e.g., BMP-2, BMP-4, BMP-6, BP-7, or BMP-2/7), or a growth factor (e.g., platelet derived growth factor (PDGF), epidermal growth factor (EGF), transforming growth factor-α (TGF-α), transforming growth factor-β (TGF-β), FGF-2, basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), or placental growth factor (PLGF). In some cases, the protein can be an interferon (e.g., interferon-α, interferon-β, interferon-λ1, interferon-λ2 or interferon-λ3). In some cases, the protein can be a thrombolytic agent (e.g., tissue plasminogen activator, antistreptase, streptokinase, or urokinase).

In some embodiments, the protein can be a hormone such as, without limitation, insulin, oxytocin, vasopressin, adrenocorticotrophic hormone, prolactin, luliberin, growth hormone, growth hormone releasing factor, parathyroid hormone, somatostatin, glucagon, interferon, gastrin, tetragastrin, pentagastrin, urogastroine, secretin, prosecretin, calcitonin, angiotensin, renin, glucagon-like peptide-1, human granulocyte macrophage colony stimulating factor (GM-CSF), nerve growth factor (NGF), platelet derived growth factor (PDGF), fibroblast growth factor (FGF), calcitonin, cortistatin, endothelin, erythropoietin, gastrin, ghrelin, inhibin, osteocalcin, luteinizing hormone, oxytocin, prolactin, secretin, renin, somatostatin, thrombopoietin, and insulin.

In some cases, the protein can be a viral antigen (e.g., an antigen derived from a virus such as adenovirus, arbovirus, astrovirus, coronavirus, Coxsackievirus, Crimean-Congo hemorrhagic fever virus, cytomegalovirus (CMV), dengue virus, ebola virus, Epstein-Barr virus (EBV), foot-and-mouth disease virus, Guanarito virus, Hendra virus, herpes simplex virus-type 1 (HSV-1), herpes simplex virus-type 2 (HSV-2), human herpesvirus-type 6 (HHV-6), human herpesvirus-type 8 (HHV-8), hepatitis A virus (HAV), hepatitis B virus (HBV), hepatitis C virus (HCV), hepatitis D virus (HDV), hepatitis E virus (HEV), human immunodeficiency virus (HIV), influenza virus, Japanese encephalitis virus, Junin virus, Lassa virus, Machupo virus, Marburg virus, Norovirus, Norwalk virus, human papillomavirus (HPV), parainfluenza virus, parvovirus, poliovirus, rabies virus, respiratory syncytial virus (RSV), rhinovirus, rotavirus, rubella virus, Sabia virus, severe acute respiratory syndrome virus (SARS), varicella zoster virus, variola virus, West Nile virus, or yellow fever virus), a parasite antigen (e.g., an antigen derived from a parasite such as Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, or Trypanosoma cruzi), or a bacterial antigen (e.g., an antigen derived from a bacterium such as Bacillus anthracis, Bordetella pertussis, Campylobacter jejuni, Chlamydia pneumoniae, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli O157:H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis).

In some embodiments, a suitable protein can be are selected from the group consisting of amyloid β peptide (Aβ), α-synuclein, microtubule-associated protein tau (Tau protein), TDP-43, Fused in sarcoma (FUS) protein, superoxide dismutase, C9ORF72, ubiquilin-2 (UBQLN2), ABri, ADan, Cystatin C, Notch3, Glial fibrillary acidic protein (GFAP), Seipin, transthyretin, serpins, amyloid A protein, islet amyloid polypeptide (IAPP; amylin), medin (lactadherin), apolipoprotein AI, apolipoprotein AII, apolipoprotein AIV, Gelsolin, lysozyme, fibrinogen, beta-2 microglobulin, crystallin, rhodopsin, calcitonin, atrial natriuretic factor, prolactin, keratoepithelin, keratin, keratin intermediate filament protein, lactoferrin, surfactant protein C (SP-C), odontogenic ameloblast-associated protein, semenogelin 1, apolipoprotein C2 (ApoC2), apolipoprotein C3 (ApoC3), leukocyte chemotactic factor-2 (Lect2), galectin-7 (Gall), corneodesmosin, enfuvirtide, cystic fibrosis transmembrane conductance regulator (CFTR) protein, and hemoglobin.

Methods for coupling a polypeptide to a functional group on a solid support include those described herein and those known in the art. Binding can be irreversible or reversible. Irreversible coupling can be accomplished by using NHS-beads or beads functionalized with aldehyde groups, for example; both of these can react with amines to form irreversible bonds. To achieve reversible coupling, a polypeptide can be bound to a solid support (e.g., an agarose bead) via a bond generated between a disulfide group on the solid support and a free thiol group on an amino acid side chain, or via a bond generated between a maleic anhydride (e.g., DMA) group on the solid support and a free amino group on the polypeptide, either at the N-terminus of the polypeptide or on an amino acid side chain. As described in the Examples below, given the pKa difference between the N-terminal amine and the side chain amines of a polypeptide, specific coupling via the N-terminus can be achieved by modulating the pH at which the reaction takes place. For example, a coupling reaction carried out at a pH between about 5.5 and about 6.5 can result in specific coupling via the N-terminal amine, whereas a reaction carried out at a higher pH (e.g., between about 6.5 and about 8) can result in coupling at the N-terminal amine and at the amino groups of lysine side chains.

After a polypeptide is bound to the solid support, it can be modified by addition of polymer subunits (e.g., monomers) to generate a polypeptide-polymer conjugate. The conjugates described herein can be generated using polymerization processes that include polymerizing monomers under controlled polymerization methods onto a polypeptide immobilized on a solid support. In general, two methods can be utilized to form polymeric chains extending from a polypeptide: a “grafting-from” approach and a “grafting-to” approach. “Grafting-to” approaches include first generating a polymer chain, and then covalently bonding the polymer chain to the surface of the protein via a polymerization initiator. In contrast, “grafting-from” approaches involve successively polymerizing a plurality of monomers onto a polymerization initiator on the surface of a polypeptide, eventually resulting in a polymeric chain being covalently bonded to the initiator. In these methods, polymerization of the polymeric chain can be conducted through free radical polymerization, such as atom transfer radical polymerization (ATRP). In some cases, other controlled radical polymerization techniques can be used.

In some embodiments, the polymeric chain can be deactivated once it reaches a desired length, to prevent further polymerization thereon. For example, if a “grafting-from” method is utilized to generate the protein-polymer conjugate (e.g., via ATRP), a deactivation agent can be attached to the end of each polymeric chain, to inhibit further polymerization thereon. Suitable deactivation agents can be selected based on the type of polymerization and/or the type(s) of monomers utilized. Deactivation agents include, without limitation, amines, peroxides, or mixtures thereof. If a “grafting-to” approach is used, the polymeric chain can be deactivated either prior to or after covalently bonding the polymeric chain to a polymerization initiator. In general, initiators are chemical species having a transferable atom that can interact with a transition metal and a ligand to form a partially soluble transition metal complex, which then can participate in a reversible redox reaction with the added initiator or a dormant polymer to form an active species to copolymerize radically polymerizable monomers.

As noted above, in some cases, protein-polymer conjugates can be generated using ATRP. In ATRP, polymerization control is achieved through an activation-deactivation process, in which most of the reaction species are in dormant format, thus significantly reducing chain termination reaction. The four major components of ATRP include a monomer, an initiator, a ligand, and a catalyst. The catalyst can determine the equilibrium constant between the active and dormant species during polymerization, leading to control of the polymerization rate and the equilibrium constant. The deactivation of radicals in ATRP can include reversible atom or group transfer that can be catalyzed by transition-metal complexes (e.g., transition metal complexes of Cu, Fe, Ru, Ni, or Os). An initiator (e.g., an alkyl halide, such as an alkyl bromide) can be activated by a transition metal complex to generate a radical species. Monomers then can be reacted with the radical species to attach a monomer to a polypeptide of interest. The attached monomer then can be activated to form another radical, and the process can be repeated with additional monomers to result in the generation of polymerized species. ATRP methods and improvements thereto are described elsewhere (see, e.g., U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166; 7,125,938; 7,157,530; 7,332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,865,797; 8,445,610; 8,871,831; 8,962,764; 9,664,042; U.S. Publication Nos. 2012/0213986; 2013/0131278; 2016/0200840; and 2017/0113934; International Publication Nos. WO 2016/130677, and WO 2015/051326; Matyjaszewski et al. ACS Symp. Ser. 685, 258-83 (1998); ACS Symp. Ser. 713, 96-112 (1998); ACS Symp. Ser. 729, 270-283 (2000); ACS Symp. Ser. 765, 52-71 (2000); ACS Symp. Ser. 768, 2-26 (2000); ACS Symposium Series 854, 2-9 (2003); ACS Symp. Ser. 1023, 3-13 (2009); ACS Symp. Ser. 1100, 1 (2012); Chem. Rev. 101, 2921-2990 (2001); and Progress in Polymer Science 32(1): 93-146 (2007), the contents of which are incorporated herein by reference in their entirety.

The protein-polymer conjugates generated using the methods and materials described herein can include polymers of any suitable length. In some cases, for example, the polymers generated on a polypeptide can have a length ranging from at least 2 monomer repeats to about 1000 monomer repeats. Thus, the polymer attached to a polypeptide can have length of about 5 to about 750 monomer repeats, about 10 to about 200 monomer repeats, about 10 monomer repeats to about 600 monomer repeats, about 25 monomer repeats to about 500 monomer repeats, about 50 monomer repeats to about 400 monomer repeats, about 100 to about 250 monomer repeats, or any other range subsumed within about 2 to about 1000 polymer repeats. In some embodiments, the polymer length can be from at least about 5 monomer repeats to about 150 monomer repeats, or from at least about 5 monomer repeats to about 200 monomer repeats.

In some cases, a polypeptide-polymer conjugate generated as described herein can include a co-polymer with more than one monomeric repeating unit. For example, a protein-polymer conjugate can include at least one polymer that is a co-polymer containing at least two different monomers. In some embodiments, the co-polymer can include at least two different monomers, where at least one monomer has topology that varies from that of at least one other monomer of the co-polymer. More specifically, the varied topology of the at least one monomer can include block, random, star, end-functional, or in-chain functional co-polymer topology. In some cases, at least one monomer of the co-polymer can include at least one monomer of a di-block topology. The co-polymers, monomers for di-block formation, monomers including an end functional group, or in-chain functional copolymers can be obtained commercially, or can be synthesized using materials and methods described elsewhere (see, e.g., U.S. Pat. Nos. 5,789,487 and 6,624,263, U.S. Publication No. 2009/0171024, and Matyjaszewski and Davis, ed., Handbook of Radical Polymerization, John Wiley and Sons, Inc., Hoboken, N.J. (2002), the entire contents of which are incorporated herein by reference.

In general, when ATRP is used to generate a protein-polymer conjugate as described herein, any accessible amino group on the unconjugated protein surface can be modified to grow a polymer. Thus, the polypeptide components of the conjugates provided herein can have a plurality of polymers coupled thereto. As such, a protein-polymer conjugate can include at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 125, at least 150, at least 175, at least 200, or more polymers.

In some embodiments, each polymer of the plurality of polymers on a polypeptide can include monomeric units of the same type. In some embodiments, however, the plurality of polymers can include a first polymer and a second polymer that are composed of monomeric units of a different type. In some cases, the plurality of polymers can include at least two different polymers (e.g., 2, 3, 4, 5, 6, 7, 8, 9, 10, 15, 20, or more than 20 polymers), each made of monomeric units of a different type. In some cases, therefore the plurality of polymers can include a first type of polymer and a second type of polymer, where the first type of polymer and the second type of polymer are each made of monomeric units of a different type. In some cases, the plurality of polymers can include at least two types of polymers, where each polymer is composed of a different combination of monomeric units. In some embodiments, for example, the plurality of polymers can include a combination of at least one positively-charged polymer and at least one zwitterionic polymer. In some cases, the plurality of polymers can include at least two positively-charged polymers, or at least two zwitterionic polymers. The monomer units of the polymers that are grown on support-coupled proteins as described herein can be from monomer classes such as, without limitation, methacrylates, acrylates, acrylamides, styrenics, acrylamide-styrenics, and combinations thereof.

If the protein was reversibly coupled to the solid support, it can be released from the support after the polymer has been assembled on the protein. For example, in some cases a protein-polymer conjugate generated according to the methods provided herein can be released from the solid support by washing at a relatively low pH (e.g., pH 3 or pH 4). Such a method can be effective for releasing proteins that were bound via an amine group, for example. In such embodiments, the protein-polymer conjugates can be resistant to acidic environments. For example, the protein-polymer conjugates described herein can be resistant to denaturation and deactivation in an environment having a pH of about 3.0 (e.g., a pH of 2.5, 2.75, 2.9, 3.0, 3.1, 3.25, or 3.5). In some cases, if a protein was attached to a solid support via a thiol group, the protein-polymer conjugate can be released from the solid support by addition of a reducing agent (e.g., dithiothreitol). If a protein was coupled to an affinity-based solid support, the protein-polymer conjugate can be released by, for example, washing with a reagent such as imidazole or ethylenediamenetetraacetic acid (EDTA), by addition of excess ligand, or by reducing the pH (e.g., to about 2.0 to 3.0).

The methods provided herein can optionally include the steps of attaching a protein to a solid support (e.g., a surface-modified solid support) that has functional groups or other moieties (e.g., ligands, in the case of an affinity resin) to which the protein can bind, adding an initiator, and then adding polymer subunits (e.g., monomers) that can successively attach to the initiators on the protein to generate polymers. In some cases, the methods can include modifying the surface of the solid support to include the desired functional groups or other moieties to achieve protein binding. In addition, the methods can include washing the solid support after modification to add functional groups and/or after addition of the protein, and washing the solid support-protein complex after addition of the initiator and/or the polymer subunits. The methods also may include additional steps, such as monitoring the size of the protein-polymer conjugate, releasing the conjugate from the solid support, and purifying the protein-conjugate after release.

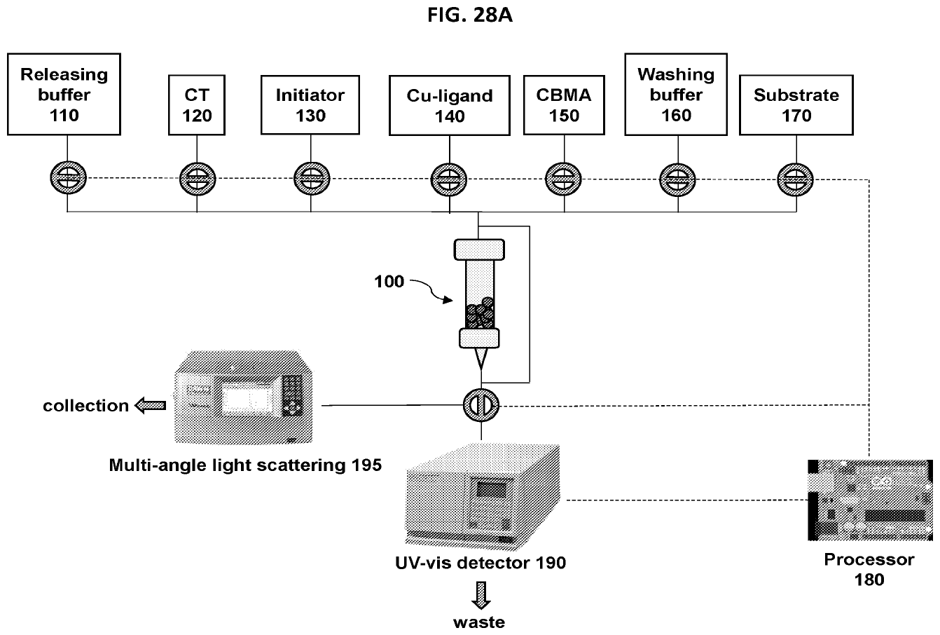

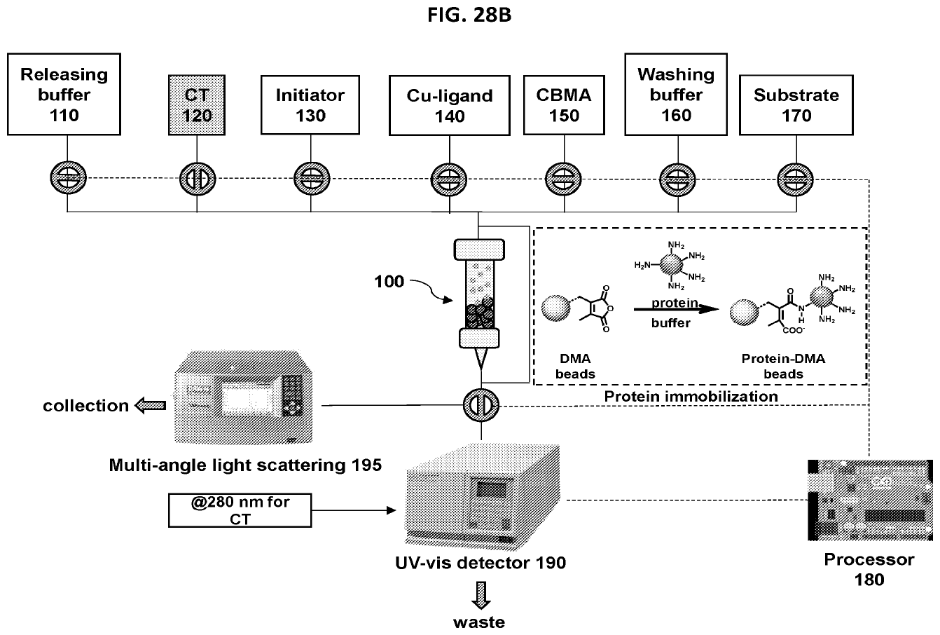

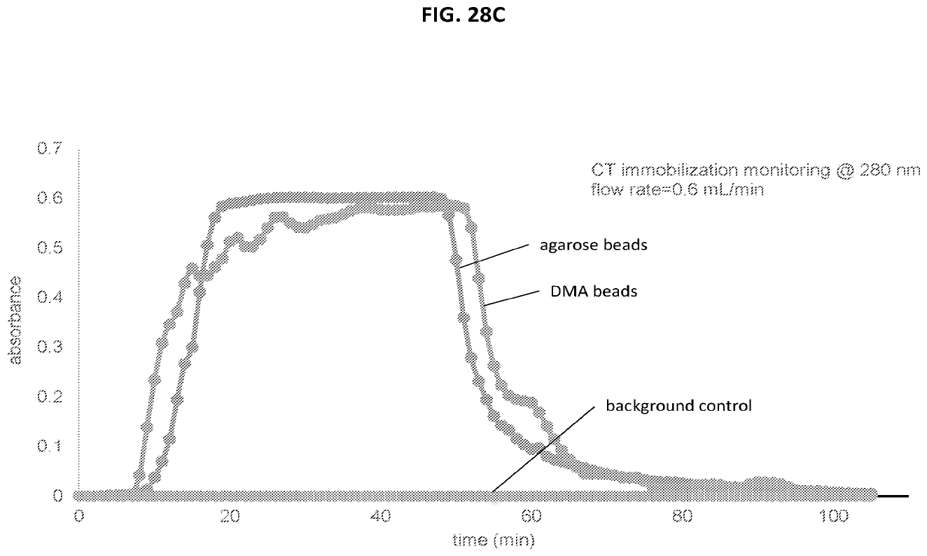

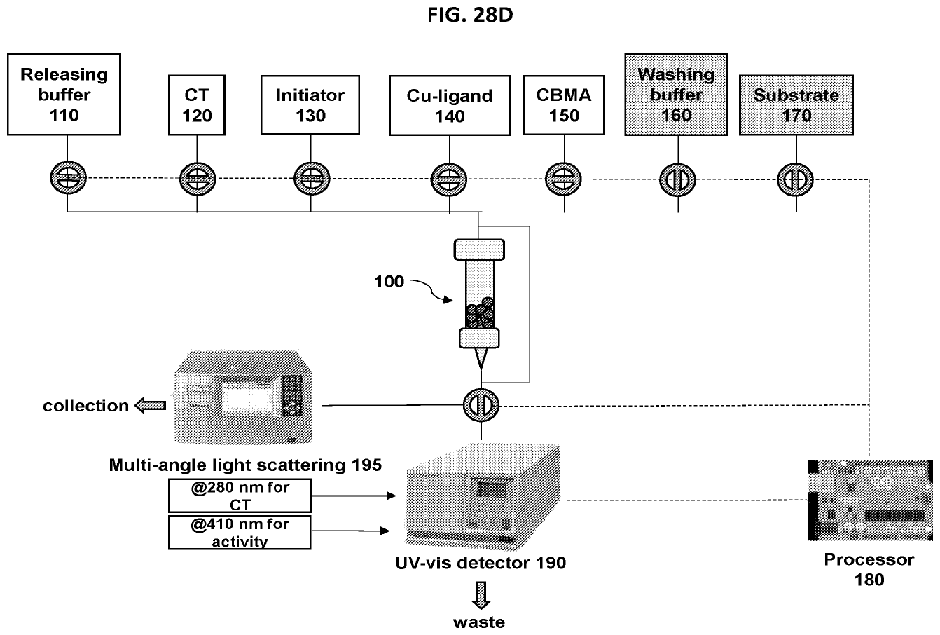

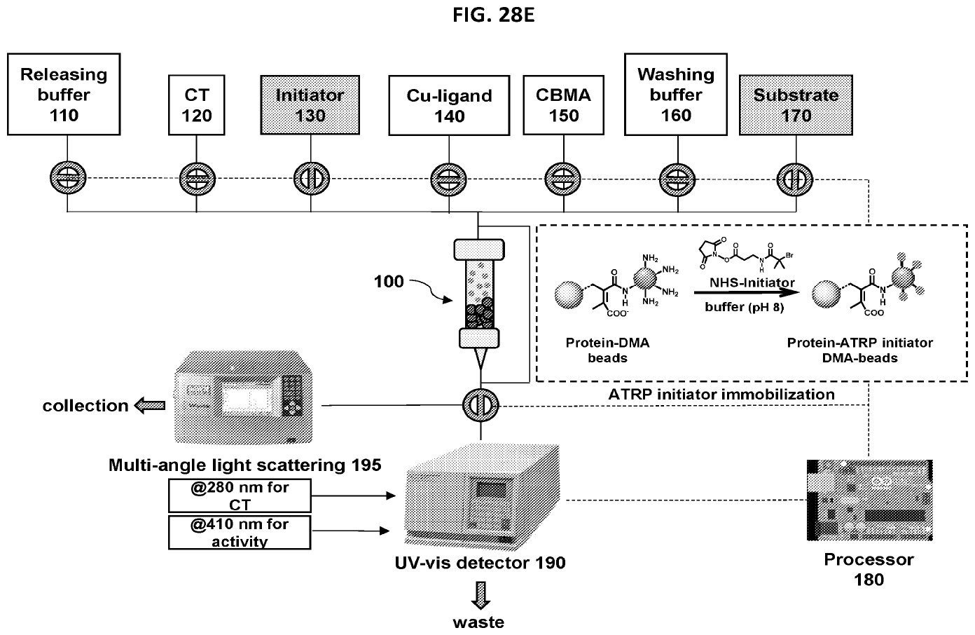

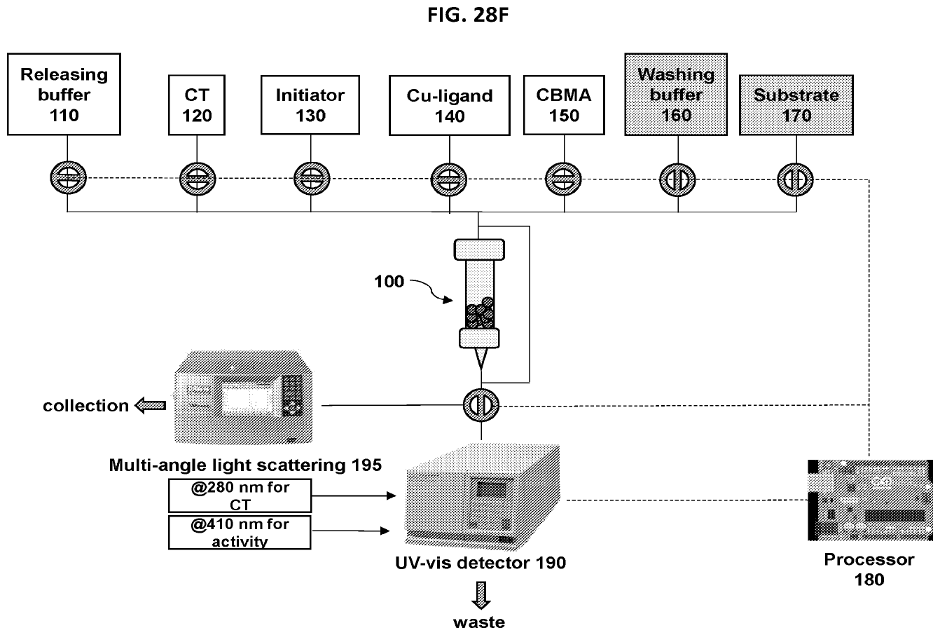

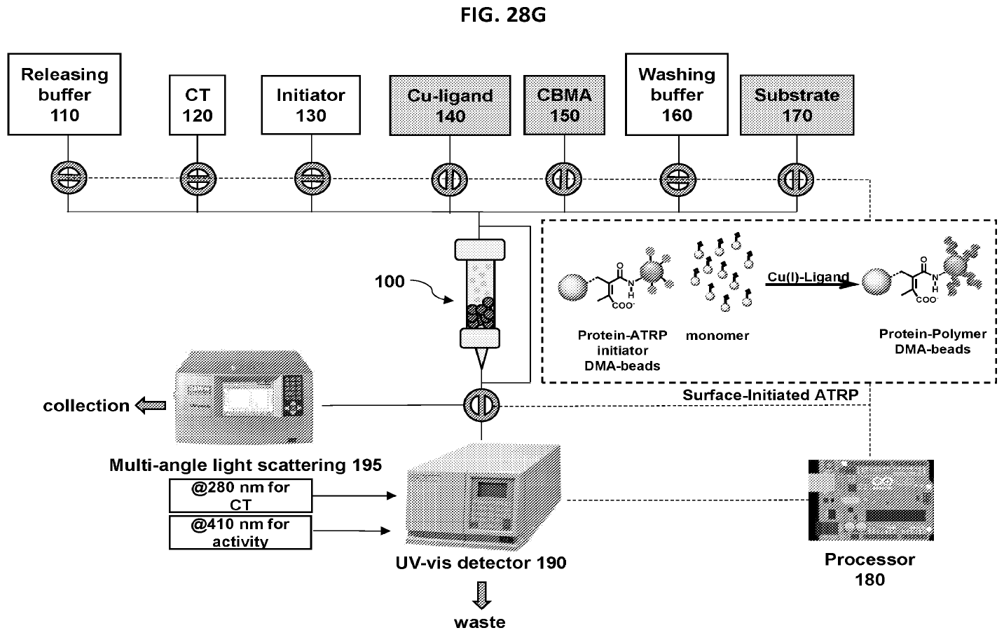

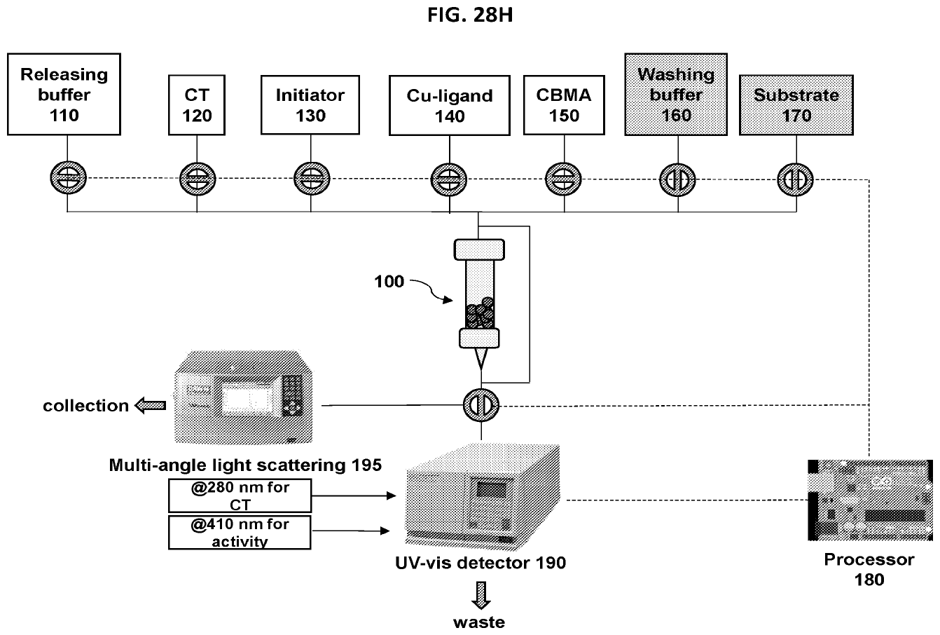

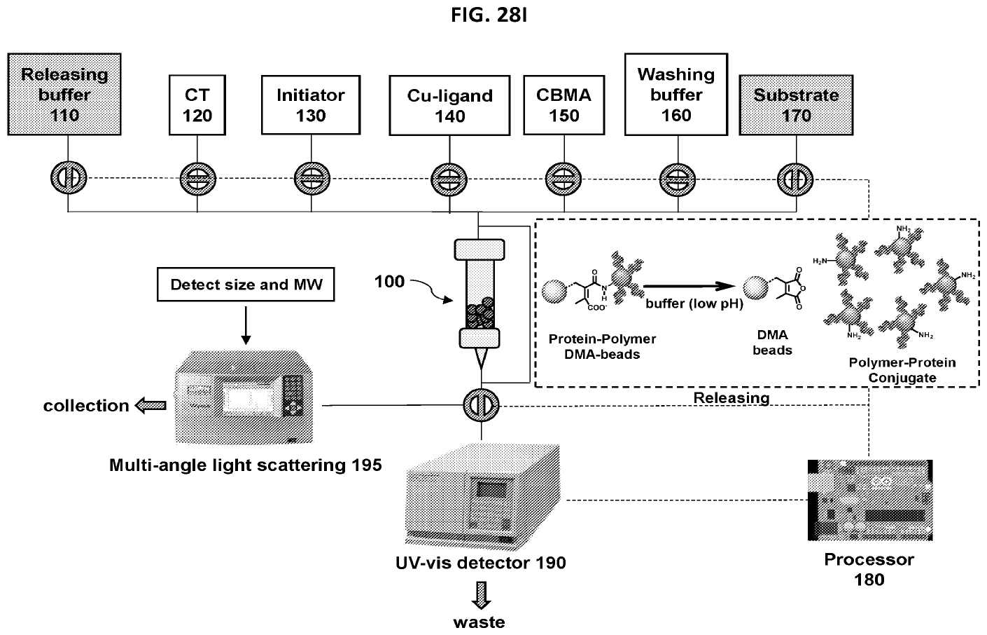

It is noted that the methods provided herein can be fully or partially automated. An exemplary scheme for an automated method is illustrated in FIGS. 28A-28I. As shown, an automated system can include column 100 that contains a solid support adapted for coupling to a protein (e.g., CT 120). Column 100 can be in fluid communication with reservoirs containing initiator 130, ligand 140, polymer (in this case, CBMA 150), washing buffer 160, substrate 170, and releasing buffer 170, all of which can be under the control of processor 180. Solutions containing the various components can be controlled to flow from their respective containers into column 100, and over the solid support at particular rates. After exiting column 100, the fluid can be monitored (e.g., by measuring UV/vis absorbance) and either collected or sent to waste. Thus, as depicted in the figures, UV-vis detector 190 and size monitoring instrument 195 can be used to monitor output from the solid support. FIG. 28A shows the overall set-up for the exemplary system. FIG. 28B depicts addition of CT protein to solid support-containing column 100, and indicates that CT immobilization on the solid support is determined based absorbance at 280 nm. The results of monitoring can be plotted as shown in FIG. 28C, for example. FIG. 28D depicts addition of wash buffer 160 and substrate 170 to the column, in order to remove unbound CT and to prevent the CT active site from becoming modified by initiator in the next step. FIG. 28E depicts addition of initiator 130 to the immobilized protein on the solid support, along with additional substrate 170. FIG. 28F depicts another wash of column 100 with wash buffer 160 and substrate 170, in order to remove excess initiator and prevent binding of polymer subunits to the active site in the next step. FIG. 28G depicts addition of ligand 140, polymer subunit 150 (CBMA), and further substrate 170 to column 100, to promote polymer formation. FIG. 28H shows addition of further washing buffer 160 and substrate 170 to remove excess ligand and polymer. Finally, FIG. 28I depicts removal of the protein polymer conjugate from solid support-containing column 100 by releasing buffer 110. The size and molecular weight of the conjugate then can be assessed using instrument 195. As described in the Examples below, the use of such a system resulted in rapid, facile production of a desired protein-polymer complex.

This document further provides the solid supports and solid support-protein complexes used in the methods provided herein, as well as protein-polymer conjugates generated by the methods provided herein. Thus, in some embodiments, this document provides a solid support displaying a functional group that is capable of reversible coupling to a polypeptide, where the solid support is a hydrophilic bead or resin, and where the functional group includes a maleic anhydride (e.g., DMA). In some embodiments, this document also provides a complex that includes a solid support reversibly coupled to a polypeptide, where the polypeptide is reversibly coupled to the solid support via a covalent bond between a dialkyl maleic anhydride group on the solid support and the N-terminal amino group of the polypeptide. Further, this document provides a protein-polymer conjugate generated according to a method provided herein. The conjugate can include a polypeptide having a plurality of polymer units covalently attached to it, where the polymer units are attached to the polypeptide via amino groups of amino acid side chains, but not via the N-terminal amino group of the polypeptide.

This document also provides compositions containing a protein-polymer conjugate generated as described herein, in combination with a pharmaceutically acceptable carrier (e.g., sterile water, saline, or a buffer). Pharmaceutical compositions can be prepared according to any method known to the art for the manufacture of pharmaceuticals, and, in some cases, can include one or more sweetening agents, flavoring agents, coloring agents, and/or preserving agents. In some cases, a pharmaceutical composition can be admixed with a nontoxic, pharmaceutically acceptable excipient. Formulations can include one or more diluents, emulsifiers, preservatives, or buffers, and can be provided in forms such as liquids, powders, emulsions, lyophilized powders, sprays, creams, lotions, controlled release formulations, tablets, pills, gels, etc., for oral, intravenous, intramuscular, subcutaneous, or other routes of administration.

Pharmaceutical formulations for oral administration can be formulated using pharmaceutically acceptable carriers that enable the pharmaceuticals to be formulated in unit dosage forms such as tablets, pills, powder, dragees, capsules, liquids, lozenges, gels, syrups, slurries, or suspensions that are suitable for ingestion by a subject. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients include carbohydrate or protein fillers include, e.g., sugars such as lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents can be added, such as cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the protein-polymer conjugates can be dissolved or suspended in suitable liquids, such as fatty oils, liquid paraffin, or liquid polyethylene glycol, with or without stabilizers.

Aqueous suspensions can contain a protein-polymer conjugate in admixture an excipients suitable for the manufacture of aqueous suspensions, e.g., for aqueous intradermal injections. Such excipients include, without limitation, suspending agents such as sodium carboxymethylcellulose, methylcellulose, hydroxypropylmethylcellulose, sodium alginate, polyvinylpyrrolidone, gum tragacanth, and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). An aqueous suspension also can contain one or more preservatives such as ethyl or n-propyl p-hydroxybenzoate, one or more coloring agents, one or more flavoring agents, and/or one or more sweetening agents, such as sucrose, aspartame, or saccharin. Formulations can be adjusted for osmolarity.

The invention will be further described in the following examples, which do not limit the scope of the invention described in the claims.

### EXAMPLES

### Example 1—Materials and Methods

Materials.

α-Chymotrypsin (CT) from bovine pancreas (type II), lysozyme from chicken egg white, acetylcholinesterase from Electrophorus electricus (electric eel, type VI-S), uricase from porcine liver (type V), and agarase from Pseudomonas atlantica were purchased from Sigma Aldrich (St Louis, Mo.). Avidin from Gallus gallus egg white was purchased from Lee Biosolutions (Maryland Heights, Mo.). Protein surface active ATRP initiator (NHS-ATRP initiator) was prepared as described elsewhere (Murata et al., supra).

Preparation of dialkyl maleic anhydride agarose (DMA) beads.

All materials were purchased from Sigma Aldrich and used without further purification unless stated otherwise. Aminated agarose beads (ω-Aminohexyl-SEPHAROSE® 4B, 10 mL, swollen, 7-12 μmol NH2/mL beads) were washed with deionized water (30 ml×2), 20 mM citric acid (30 ml×2) and 100 mM sodium phosphate buffer (pH 9, 30 mL×2). A solution of 2,5-dihydro-4-methyl-2,5-dioxo-3-furanpropanoic acid (44 mg, 240 μmol, TCI America, Philadelphia, Pa.), 1-ethyl-3-(3-dimethylaminopropyl)carbodiimide.HCl (EDC.HCl) (46 mg, 240 μmol) and 1-hydroxybenzotriazole hydrate (32 mg, 240 μmol) was pre-incubated in dimethylformamide at 0° C. for 30 minutes and then added to the aminated agarose solution with trimethylamine (70 μL, 500 μmop, and the mixture was shaken at room temperature for 30 minutes. The beads were washed with deionized water (30 mL×3), 20 mM citric acid (30 mL×3) and deionized water (30 mL×2). To block unmodified amine groups, the beads were incubated with acetic anhydride (71 μL, 500 μmop and trimethylamine (70 μL, 500 μmol) in deionized water (30 mL) at room temperature for 30 minutes. The beads were then washed as described above and stored in the refrigerator.

Quantitative analysis of accessible DMA groups on agarose beads.

Prepared DMA beads (10 μL) were placed in a solution of 40 μM Cyanine3 amine (Lumiprobe, Hallandale Beach, Fla.) in 100 mM sodium phosphate buffer (pH 8) containing 0.05 v/v % TWEEN® 20 (500 μL) and shaken at room temperature for 60 minutes. Beads were settled by centrifugation and the supernatant was aspirated. The beads were then washed with 100 mM sodium phosphate (pH 8) containing 0.05 v/v % TWEEN® 20 (1 mL×5). After removing the supernatant, 20 mM sodium citrate (pH 3) containing 0.05 v/v % TWEEN® 20 (1 mL) was added to the beads and the mixture was shaken at room temperature for 60 minutes. Supernatant fluorescence intensities from the releasing solution were measured at an excitation of 550 nm and an emission of 570 nm with 10 nm bandwidths by a SAFIRE2™ plate reader (Tecan, Group Ltd.). Concentrations were calculated from standard curves.

Preparation of Glycyl-glycyl-Cy3 (GGCy3).

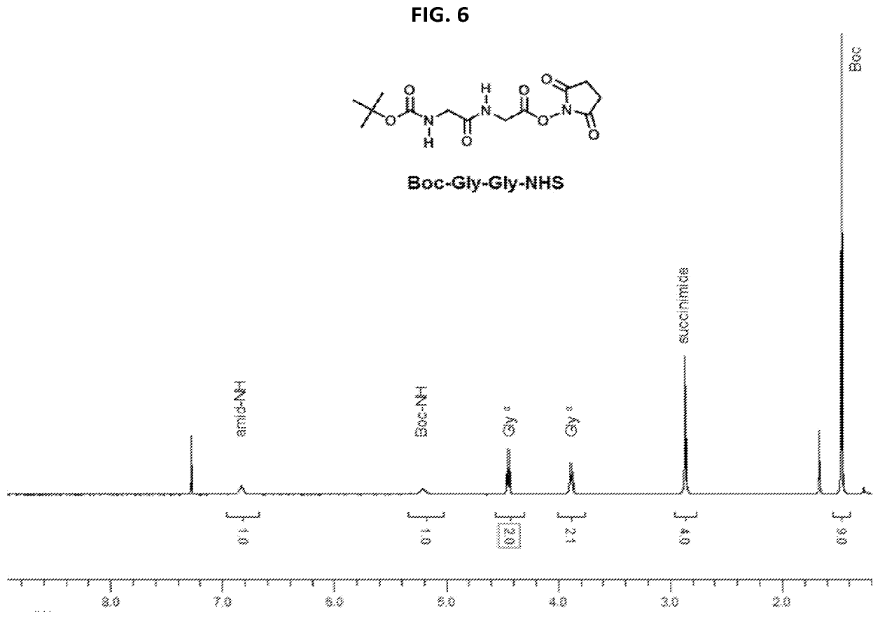

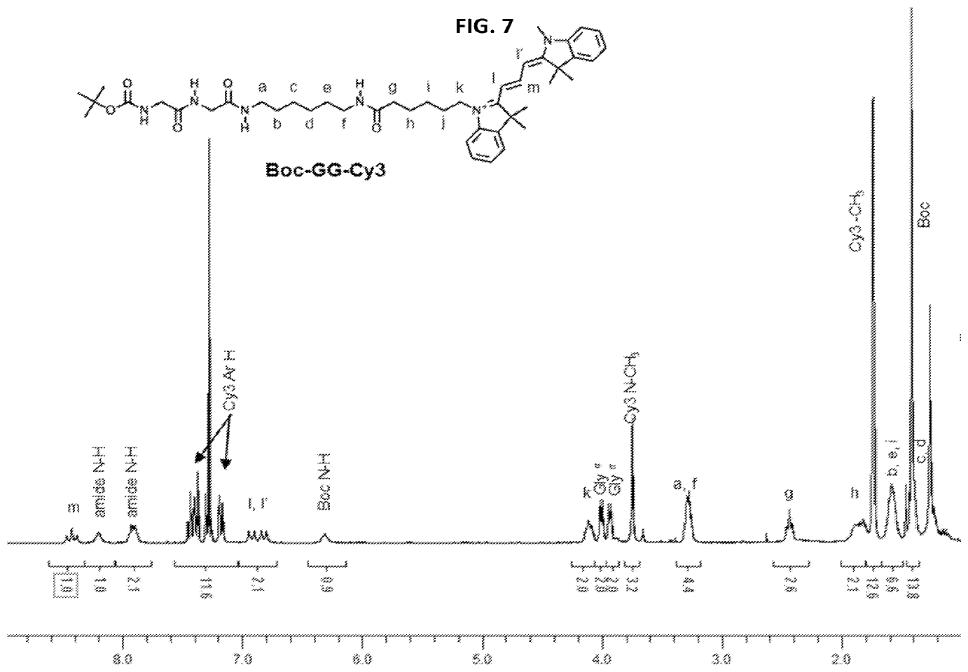

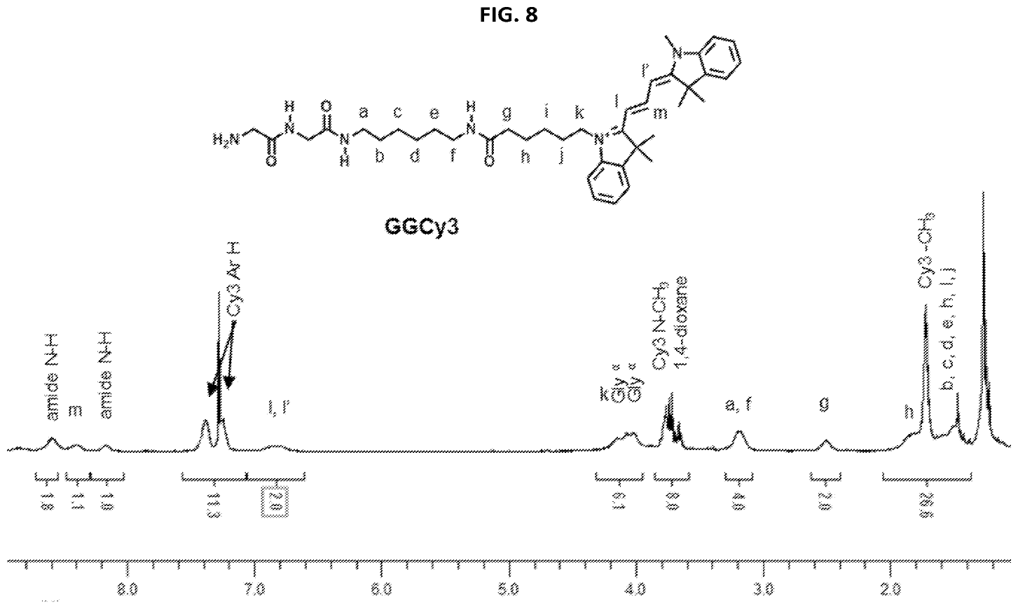

N,N′-Diisopropylcarbodiimide (770 μL, 5.0 mmol) was added to a solution of Boc-GlyGly-OH (Bachem America, Torrance, Calif.; 920 mg, 4.0 mmol) and N-hydroxysuccinimide (NHS, 575 mg, 5.0 mmol) in dichloromethane (50 mL) at 0° C. The solution was stirred at room temperature overnight. Precipitated urea was filtered out, and the filtrate was evaporated to remove dichloromethane under vacuum. Boc-GlyGly-NHS was isolated by recrystallization in 2-propanol with a 63% yield verified with proton nuclear magnetic resonance spectroscopy (FIGS. 6-8) recorded in CDCl₃ using a 300 MHz, Bruker Avance. The solution of Boc-GlyGly-NHS (16 mg, 48 μmol) in chloroform (40 mL) was added to a solution of Cyanine3 amine (25 mg, 40 μmol) and trimethylamine (7 μL, 50 μmol) in DMSO (100 μL) and stirred at room temperature overnight. The mixture was washed with 0.5 N HCl aq. (50 mL×2), saturated NaHCO₃ (50 mL×2), and saturated NaCl (50 mL×2), then dried with MgSO₄. After MgSO₄ removal by filtration, Boc-GlyGly-Cy3 was isolated by evaporating chloroform under vacuum with an 89% yield verified by ¹H NMR. The mixture of Boc-GlyGly-Cy3 (30 mg, 35.6 μmol) in 4 M hydrochloric acid (HCl), 1,4-dioxane (80 μL), and 1,4-dioxane (920 μL) was stirred at room temperature for 4 hours. GlyGlyCy3 was obtained by evaporation of dioxane under vacuum, with a 98% yield verified with ¹H NMR.

GGCy3, oily compound, ¹H NMR (300 MHz, CDCl₃) δ 1.4-1.9 (broad, 14H, 7×CH₂ and 12H, Cy3-CH₃), 2.5 (broad, 2H, CH₂), 3.2 (broad, 4H, 2×CH₂), 3.8 (broad, 3H, Cy3 N—CH₃), 4.0-4.3 (broad, 2H, CH₂ and 4H, 2×Gly^{α}), 6.8 (broad, 2H, CH═CH), 7.3 and 7.4 (broad, 8H Cy3-Ar H), 8.2 (broad, 1H, amide), 8.4 (broad, 1H, CH═CH—CH), 8.6 (broad, 2H, amide) ppm; ¹³C NMR (75 MHz, DMSO-d₆) δ 25.2, 25.7, 26.1, 26.8, 28.0, 28.2, 28.7, 28.9, 29.0, 29.1, 29.7, 34.0, 35.0, 36.1, 38.7, 39.7, 42.9, 49.0, 49.2, 103.9, 105.6, 116.0, 125.7, 127.5, 128.5, 131.2, 140.5, 141.9, 169.3, 170.1, 173.8 ppm; IR (NaCl plate) 2956, 2924, 2853, 1712, 1651, 1557, 1493, 1456, 1416 and 1376 cm⁻¹; HRMS (m/z): [M−2H]⁺ calcd. for C₄₀H₅₇N₆O₃²⁺, 670.46; found, 670.94.

GGCy3 and Cy5.5 amine binding to DMA beads.

500 μL of GGCy3 or Cy5.5 amine solution (Lumiprobe, Hallandale Beach, Fla.), 40 μM in 100 mM sodium phosphate (pH 5-8) containing 0.05 v/v % TWEEN® 20 and 20 μL of DMA beads were shaken at room temperature. The supernatant was removed at given incubation times and the beads were washed with incubation buffer (1 mL×2) followed by washing buffer (100 mM sodium phosphate (pH 8) containing 0.05 v/v % of Tween 20 (1 mL×3) to remove residuals. The beads were incubated in releasing buffer (1 mL, 20 mM sodium citrate (pH 3) containing 0.05 v % Tween 20) at room temperature for 1 hour, and supernatant fluorescence intensities were measured with a SAFIRE2™ plate reader (GGCy3: excite 550 nm, emit 570 nm; Cy5.5: excite 670 nm, emit 707 nm). Concentrations were calculated from standard curves.

GGCy3 and Cy5.5 amine release from DMA beads.

30 μL of DMA beads were pre-incubated with 750 μL of GGCy3 or Cy5.5 amine solution (40 μM in 100 mM sodium phosphate (pH 8) containing 0.05 v/v % TWEEN® 20). The supernatant was removed and the dye bound beads were washed with the washing buffer. The beads were incubated in releasing buffer (1.5 mL) at room temperature and fluorescence intensities of supernatant aliquots (100 μL) at given time points were measured at the wavelengths mentioned above.

Protein immobilization on DMA beads.

4.5 mL of protein solution (2 mg/mL, 100 mM sodium phosphate (pH 6 or 8) containing 0.05 v/v % TWEEN® 20 was combined with 1.5 mL of DMA beads in the solid phase peptide synthesis vessel (10 mL capacity, Chemglass) and shaken at room temperature or in refrigerator for 30 minutes. After removing the supernatant, the beads were washed with incubation and washing buffers. The amount of immobilized protein on the DMA beads was determined using a Micro BCA Protein Assay Kit (ThermoFisher Scientific). The sample solution and beads (10 μL) in 500 μL of deionized water were mixed with micro BCA working reagent (500 μL) and incubated at 60° C. for 1 hour. The absorbance at 562 nm was recorded by a UV-VIS spectrometer (Lambda 2, Perkin Elmer).

ATRP initiator modification onto immobilized protein.

200 mM of ATRP initiator, N-2-bromo-2-methylpropanoyl-β-alanine N′-oxysuccinimide bromide (NHS—Br; Murata et al., supra) solution in DMSO (225 μL) was added to the suspension of the protein immobilized DMA beads (1.5 mL) in 100 mM sodium phosphate (pH 8, 4.5 mL) and shaken at room temperature for 30 minutes. The beads were washed with 100 mM sodium phosphate (pH 8, 5 mL×5). Estimation of immobilized ATRP initiator on protein was carried out by BCA and fluorescamine assays. The beads (20 μL) were incubated in 20 mM sodium citrate (pH 3) containing 0.05 v/v % of TWEEN® 20 (200 μL) at room temperature for 1 hour. A BCA protein assay was used to determine protein concentration in the supernatants as described above. A fluorescamine assay was used to determine the number of bound initiators. Aliquots (40 μL) of supernatant, 100 mM sodium phosphate (40 μL, pH 9), and fluorescamine solution in DMSO (20 μL, 3 mg/mL) were added into a 96 well plate and incubated at room temperature for 15 minutes. Fluorescence intensities were measured at an excitation of 390 nm and emission of 470 nm with 10 nm bandwidths by a SAFIRE2™ plate reader. Concentrations were determined using a standard curve.

Trypsin Digestion of Protein-Initiator Conjugates.

Trypsin digests were used to generate peptide fragments from which protein-initiator attachment sites could be determined using matrix-assisted laser desorption/ionization time-of-flight (MALDI-ToF) mass spectrometry. Five proteins were studied: α-chymotrypsin (CT) from bovine pancreas (type II), lysozyme from chicken egg white, acetylcholinesterase from Electrophorus electricus (electric eel, type VI-S), and uricase from porcine liver (type V). Avidin from Gallus gallus egg white was purchased from Lee Biosolutions (Maryland Heights, Mo.). Samples were digested according to the protocol described in the In-Solution Tryptic Digestion and Guanidination Kit. Briefly, 10-20 μg of protein or protein-initiator complexes (10 μL of a 2 mg/mL protein solution in deionized water) were added to 15 μL of 50 mM ammonium bicarbonate with 1.5 μL of 100 mM dithiothreitol (DTT) in a 0.5 mL Eppendorf tube. The reaction was incubated for 5 minutes at 95° C. Thiol alkylation was conducted by the addition of 3 μL of 100 mM iodoacetamide aqueous solution to the protein solution, followed by incubation in the dark for 20 minutes at room temperature. After incubation, 1 μL of 100 ng trypsin was added to the protein solution and the reaction was incubated at 37° C. for 3 hours. An additional 1 μL of 100 ng trypsin was then added. The reaction was terminated after a total reaction time of 5 hours by the addition of trifluoroacetic acid (TFA). Digested samples were purified using ZipTipC₁₈ microtips and eluted with 2 μL of matrix solution (20 mg/mL sinapinic acid in 50% acetonitrile with 0.1% TFA) directly onto a MALDI-ToF plate for subsequent analysis. The molecular weights of the expected peptide fragments before and after digestion were predicted using PeptideCutter (ExPASy Bioinformatics Portal, Swiss Institute of Bioinformatics). Peptide fragments containing the N-terminal group were examined for modification.

MALDI-ToF analysis.

MALDI-ToF measurements were recorded using a PerSeptive Voyager STR MS with nitrogen laser (337 nm) and 20 kV accelerating voltage with a grid voltage of 90%. At least 300 laser shots covering the complete spot were accumulated for each spectrum. Sinapinic acid (20 mg/mL) in 50% acetonitrile with 0.4% trifluoroacetic acid was used as matrix. Protein solution (0.5-1.0 mg/mL) was mixed with an equal volume of matrix and 2 μL of the resulting mixture was loaded onto a silver sterling plate target. Apomyoglobin, cytochrome C, and aldolase were used as calibration samples. Extent of modification was determined by subtracting the protein-initiator conjugates m/z values from native protein m/z and dividing by the molecular weight of the initiator (220.9 g/mol). Molecular weights of peptide fragments obtained in protein digests were determined after the solutions were purified by use of ZipTipC₁₈ microtips. Bradykinin fragment, angiotensin II (human) and insulin oxidized B chain (bovine) were used for calibration.

Surface-initiated ATRP from immobilized protein.

A suspension of DMA beads (1.5 mL) and carboxybetaine methacrylate (CBMA, 29 mg, 125 μmol, TCI America) in 100 mM sodium phosphate (4.5 mL, pH 8) in the synthesis vessel was sealed with a rubber septum and bubbled with nitrogen at room temperature for 30 minutes. 500 μL of deoxygenated catalyst solution (CuC12, sodium ascorbate, and 1, 1, 4, 7, 10, 10-hexamethyltriethylenetetramine (HMTETA), similar conditions as in solution-based synthesis) was then added to the synthesis vessel under nitrogen. The mixture was sealed and shaken at room temperature for 1-2 hours. The beads were washed with 100 mM sodium phosphate (pH 8, 5 mL×5).

Protein-pCBMA releasing from DMA beads.

Agarose solution (15 μL, 1 U/μL) was added to a suspension of obtained protein-pCBMA beads (1.5 mL) in 100 mM sodium phosphate (pH 6, 985 μL) and rotated at room temperature overnight. To release, 20 mM sodium citrate (3.5 mL, pH 3) was added and rotated at room temperature for 1 hour. 100 mM sodium phosphate buffer (pH 5) was used for releasing AChE-pCBMA from beads due to irreversible inactivation of AChE at low pH. The supernatant containing protein-pCBMA conjugates was separated from the beads by filtration or centrifugation. Protein concentration in the supernatant was determined by UV absorption protein or BCA protein assay.

Native CT stability at pH 3.

Native CT (40 μM) was dissolved in 20 mM citrate buffer (pH 3) and incubated at 25° C. At given time points, aliquots (10 μL) were removed and measured activity in 950 μL of 100 mM sodium phosphate buffer (pH 8) and 40 μl, of suc-AAPF-pNA solution (10 mM in DMSO) at 25° C. The residual activity was calculated as a ratio of initial rates of hydrolysis reaction at given incubation time over the initial activity at time zero, which monitored by recording the increase in absorption at 412 nm using an UV-VIS spectrometer.

Solution-based synthesis of protein-pCBMA.

Solution-based synthesis of CT-pCBMA was carried out as described elsewhere (Murata et al., supra; and Cummings et al. 2017, supra). Briefly, a solution of CBMA (104 mg, 0.45 mmol) and protein-initiator (18-20 μmol of initiator) in 100 mM sodium phosphate (20 mL, pH 8) was sealed and bubbled with nitrogen gas in an ice bath for 30 minutes. 1 mL of deoxygenated catalyst solution (described above) was then added to the polymerization reactor under bubbling nitrogen. The mixture was sealed and stirred at room temperature for 2 hours. The conjugate was isolated by dialysis with a 25 kDa molecular weight cutoff dialysis tube in deionized water in a refrigerator for 24 hours and then lyophilized.

Characterization of PARIS by FT-IR spectroscopy.

100 μL of beads at each step of the PARIS synthesis were rinsed with deionized water (1 mL×5), and then frozen and dried under vacuum. FT-IR spectra were obtained for each sample with an IR spectrometer using a KBr pellet.

Characterization of released PARIS CT-pCBMA.

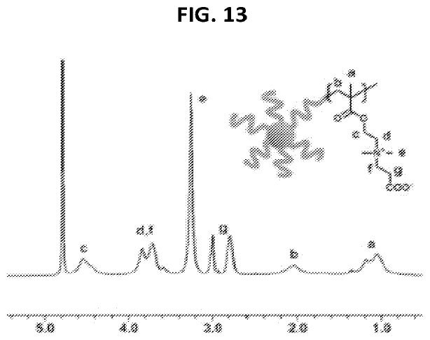

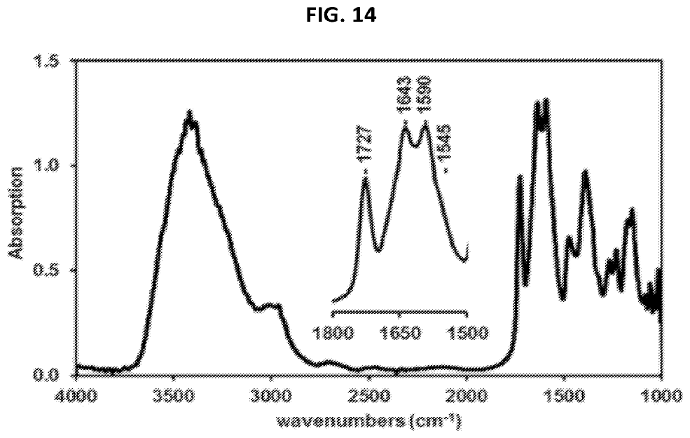

Chemical structures of the released CT-pCBMA conjugates by PARIS were characterized by ¹H NMR and FT-IR measurements. In the ¹H NMR spectrum, the signals of polymer backbone chain were observed at 1.0-1.3 (3H, methyl) and 2.1 ppm (2H, ethylene). The signals of carboxybetaine side chain (FIG. 13) were observed. In the IR spectrum, the absorption of ester group on the polymer chain was observed at 1727 cm⁻¹, and the specific absorption of amide groups from CT were observed at 1643 (ν_{C=O}: amide I) and 1550 cm-1 (δ_{N—H}: amide II).

Removal of grafted pCBMA from the conjugate.

The grafted pCBMA was removed from the conjugate by acidic hydrolysis. Protein-pCBMA conjugate (10-20 mg) and 6 N HCl aq. (4-5 mL) were placed in a hydrolysis tube. After three freeze-pump-thaw cycles, hydrolysis was performed at 110° C. for 24 hours under vacuum. The cleaved polymer was isolated by dialysis using a 1 kDa molecular weight cut off dialysis tube in deionized water, and then lyophilized. The molecular weight of the cleaved polymer was measured by GPC.

Determination of conjugate hydrodynamic diameter.

Dynamic light scattering data was collected on a Malvern Zetasizer nano-ZS. The hydrodynamic diameters of native protein and conjugate were measured three times (5 run to each measurement) in various buffers at room temperature. Reported values are number distribution intensities.

Determination of conjugate Michaelis-Menten kinetics.

Suc-AAPF-pNA (0-125 μL of 9.60 mM in DMSO) was mixed with sodium phosphate buffer (865-950 μL of 100 mM buffer, pH 8). Native CT or conjugate solutions (10 μL, 3.9 μM of CT) was added to the substrate solution. The initial substrate hydrolysis rate was monitored by recording the increase in absorbance at 412 nm using a UV-VIS absorbance spectrometer with a temperature-controlled cell holder at 25° C. Michaelis-Menten parameters were determined by nonlinear curve fitting of initial rate versus substrate concentration plots using Enzfitter software.

Determination of enzyme thermostability.

Native CT and conjugates (1.5 to 2.0 mL, 3.9 μM of CT) were incubated in 100 mM sodium phosphate buffer (pH 8.0) at 50° C. Aliquots (10 μL) were removed and activity was measured using suc-AAPF-pNA (40 μL of 9.6 mM in DMSO) in sodium phosphate buffer (950 μL of 100 mM, pH 8) by UV-VIS spectroscopy with a temperature-controlled cell holder. Residual activity was calculated as a ratio of hydrolysis rate at a given incubation time over the initial hydrolysis rate for each sample.

Flow reactor CT immobilization on DMA beads.

DMA beads (1.5 mL) and 20 mM citrate (3.0 mL, pH 2) were sealed in the flow reactor with a rubber septum. Deionized water was introduced into the reactor by a peristaltic pump at room temperature at a flow rate of 1 mL min⁻¹ for 30 minutes. CT (2.0 mg/mL) in 100 mM sodium phosphate (pH 6 or 8) containing 0.05 v/v % TWEEN® 20 was introduced into the reactor by a peristaltic pump at a flow rate of 1 mL min⁻¹ for 30 minutes to bind the DMA beads. The beads were then washed with 100 mM sodium phosphate (pH 8) containing 0.05 v/v % TWEEN® 20 for 30 minutes.

Flow reactor ATRP initiator modification on immobilized CT.

200 mM NHS—Br in DMSO was introduced into the reactor by a syringe pump at a flow rate of 8 μL min⁻¹ for 30 minutes. The sample was then washed with 100 mM sodium phosphate (pH 8) for 30 minutes. An aliquot (20 μL of beads) was taken from the reactor for the BCA and fluorescamine assays.

Flow Reactor surface-initiated ATRP from immobilized CT.

A suspension of DMA beads (1.5 mL) and CBMA (29 mg, 125 μmop in 100 mM sodium phosphate (4.5 mL, pH 8) was sealed in the synthesis vessel with a rubber septum and bubbled with nitrogen at room temperature for 30 minutes. 500 μL of deoxygenated catalyst solution (CuCl₂, sodium ascorbate, and HMTETA) was then added to the synthesis vessel under nitrogen. The mixture was sealed and stirred at room temperature for 2 hours, followed by washing with 100 mM sodium phosphate (pH 8) at a flow rate 1 mL min⁻¹ for 30 minutes.

Flow Reactor CT-pCBMA release from DMA beads.

Agarose solution (15 μL, 1 U/μL) was added to a suspension of CT-pCBMA beads (1.5 mL) in 100 mM sodium phosphate (pH 6, 985 μL) and was rotated at room temperature overnight. To release, 20 mM sodium citrate (3.5 mL, pH 3) was added and stirred at room temperature for 1 hour. The supernatant containing CT-pCBMA conjugates was separated from the beads by filtration. Protein concentration in the supernatant was determined by UV absorption and enzyme hydrolysis of N-succinyl-Ala-Ala-Pro-Phep-nitroanilide (suc-AAPF-pNA) using a standard curve with native CT.

Preparation of Cu-HMTETA as deoxygenated catalyst solution.

100 mM CuCl₂ in deionized water (1.2 mL, 120 μmol) was bubbled with N₂ for 25 minutes and then 100 mM sodium ascorbate in deionized water (120 μL, 12 μmol) was added. HMTETA (39 μL, 144 μmol) was added to the copper suspension bubbled with N₂ for 3 minutes. The deoxygenated Cu-HMTETA solution was added to the synthesis vessel immediately.

2-Nitro-5-thiocyanobenzoic (NTCB) digestion of protein-initiator conjugates.

All buffers and reagents were prepared fresh for the NTCB reactions. Native protein or initiator-modified protein complex (10-20 μg) was dissolved in 1 M glycine, 6 M guanidine-HCl pH 10.0 (15 μL) and treated with 1.5 μL of 100 mM of DTT for 5 minutes at 95° C. After this time, 22 mM NTCB (20-fold excess of total cysteine content in protein) was added and the digestion was carried out at 37° C. for 18 hours. The reaction was stopped by adding 3 μL of TFA, and digested samples were purified using ZipTipCis microtips and eluted with 2 μL of matrix solution (20 mg/mL sinapinic acid in 50% acetonitrile with 0.1% TFA) directly onto a MALDI-ToF plate for subsequent analysis. The molecular weight of the expected peptide fragments before and after digestion was predicted using PeptideCutter (ExPASy Bioinformatics Portal, Swiss Institute of Bioinformatics). Peptide fragments containing the N-terminal group were examined for modification.

Irreversible inactivation of native AChE in low pH (3-6).

Native AChE (0.4 μM) was dissolved in 20 mM citrate buffer (pH 3-6) or 100 mM sodium phosphate buffer (pH 5 and 6) and incubated at room temperature. At given time points, aliquots (10 μL) were removed and activity was measured in 930 μL of 100 mM sodium phosphate buffer (pH 7.4), 50 μL of acetylthiocholine iodide (10 mM in cold 100 mM sodium phosphate buffer (pH 7.4), Sigma Aldrich, St Louis, Mo.) and 10 μL of 5,5′-dithiobis(2-nitrobenzoic acid) (DTNB, 50 mM in DMSO, Sigma Aldrich) at 37° C. The residual activity was calculated as a ratio of initial rates of the reaction at given incubation time over initial activity of native AChE (0.4 μM) in 100 mM sodium phosphate buffer (pH 7) at time zero. Rates were monitored by recording the increasing in absorption at 412 nm using a UV-VIS spectrometer.

Determination of Michaelis-Menten kinetics of AChE-pCBMA conjugates.

Acetylthiocholine iodide (0-100 μL of 10 mM in 100 mM sodium phosphate buffer (pH 7.4)) and 10 μL of DTNB solution (50 mM in DMSO) were mixed with 100 mM sodium phosphate buffer (980-880 μL, pH 7.4). Conjugate solution (10 μL, 5.5 μM of AChE) was added to the substrate solution. The initial substrate hydrolysis rate was monitored by recording the increase in absorbance at 412 nm using a UV-VIS absorbance spectrometer with a temperature-controlled cell holder at 37° C. Michaelis-Menten parameters were determined by nonlinear curve fitting of initial rate versus substrate concentration plots using Enzfitter software.

Activity assays of Lyz-pCBMA conjugates.

Activity of Lyz-pCBMA conjugates was determined using two different substrates. Lyophilized Micrococcus lysodeikticus (Sigma Aldrich) was used to monitor enzymatic catalysis of cell wall lysis (Smolelis and Hartsell, J. Bacteriol. 58:731-736, 1949). Absorption at 450 nm of suspended M. lysodeikticus (990 μL, 0.2 mg/mL) in 50 mM phosphate buffer (pH 6.0) was measured by UV-VIS spectrometer. 10 μL of Lyz-pCBMA solution (2.8 μM in 50 mM phosphate buffer (pH 6.0)) was added and the change of absorbance at 450 nm at room temperature was monitored.

p-nitrophenyl β-glycoside of N-acetylchitooliosaccharide 2 also was used to determine the activity of the conjugates. To a solution of 4-Nitrophenyl β-D-N,N′,N″-triacetylchitotriose (10 μL of 50 mM in DMSO, Sigma Aldrich) in 50 mM phosphate buffer (980 μL, pH 6.0), conjugate solution (10 μL of 714 μM in 50 mM phosphate buffer, pH 6.0) was added and the absorption was measured at 405 nm using a UV-VIS absorbance spectrometer with a temperature-controlled cell holder at 37° C. The p-nitrophenol releasing rate was reported as hydrolysis activity of the conjugates.

Binding affinity of HABA to Avi-pCBMA conjugates.

4′-hydroxyazobenzene-2-carboxylic acid (HABA, Sigma Aldrich) is a reagent that binds to avidin and shows spectral changes, and thus it can be utilized for determination of avidin binding affinity (Green, Biochem. J. 94:23-24, 1965). Absorption at 500 nm of 300 μM HABA solution in phosphate buffered saline without calcium or magnesium (986 μL, Lonza) was measured using UV-VIS spectrometer. 16 μL, of the conjugates solution (1.25 μM of avidin in deionized water) was added to the HABA solution and incubated at room temperature for 1 minute, and then absorption at 500 nm was measured. Change in absorbance at 500 nm was used to determine bound HABA to the conjugate.

Activity assay of Uox-pCBMA.

Enzymatic activity of the Uox-pCBMA conjugates was determined by oxidation of uric acid to allantoin (Mahler et al., J. Biol. Chem. 216:625-641, 1955). Absorption at 290 nm of 50 μM uric acid in 20 mM sodium borate buffer (pH 8.5, 990 μL) was measured using a UV-VIS absorbance spectrometer with a temperature-controlled cell holder at 37° C. Conjugate solution (10 μL, 57 μM of Uox in 20 mM borate buffer (pH 8.5)) was added to the substrate solution. The initial reaction velocity was monitored by recording the decrease in absorbance at 290 nm using the UV-VIS absorbance spectrometer at 37° C. Activity of the conjugates (U/g) was determined from the initial velocity and concentration of Uox.

Impact of agarase incubation for CT-pCBMA releasing from DMA beads.

To a suspension of obtained CT-pCBMA beads (100 μL) in 100 mM sodium phosphate (pH 6, 99 μL), agarose solution (1 μL, 1 U/μL) was added and rotated at room temperature for a given time (0 to 24 hours). 20 mM sodium citrate (400 μL, pH 3) was added and rotated at room temperature for 1 hour. The supernatant containing CT-pCBMA conjugate was separated from the beads by centrifugation. Released active CT concentration in the supernatant was determined by an enzymatic activity assay on the hydrolysis of suc-AAPF-pNA using a standard curve with native CT.

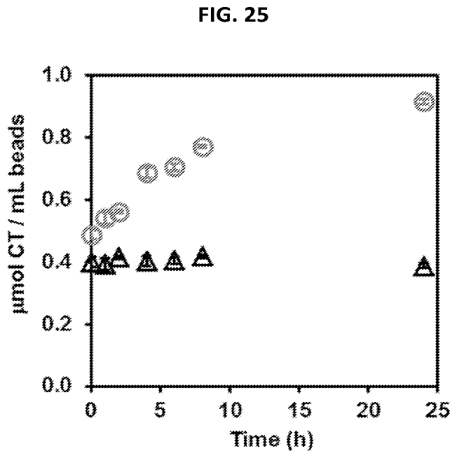

A release study of CT-pCBMA was carried out by pre-incubation with agarase to digest agarose (Yaphe, Can. J. Microbiol. 3:987-993, 1957) for quantitative recovery of the conjugate from the DMA beads. CT-pCBMA that was previously prepared on the DMA beads was pre-incubated with agarase (1 U/100 μL beads) in 100 mM sodium phosphate (pH 6) at room temperature for a designated time, followed by incubation with 20 mM sodium citrate (pH 3). Released conjugates were quantified by an activity assay of hydrolysis of suc-AAPF-pNA. Agarase pre-incubation increased recovery of the conjugate with incubation time (FIG. 25). The agarase pre-incubation (1 U/100 μL of beads) in 100 mM sodium phosphate (pH 6) at room temperature overnight before adding 20 mM sodium citrate (pH 3) was adopted for releasing the conjugate from DMA beads.

### Example 2—PARIS Chemistry

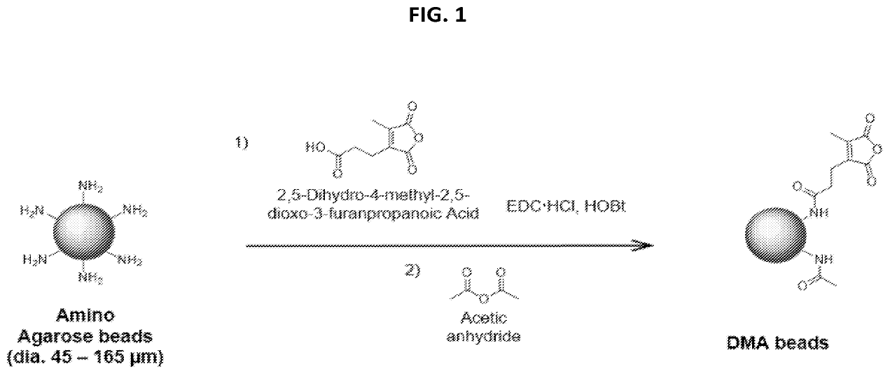

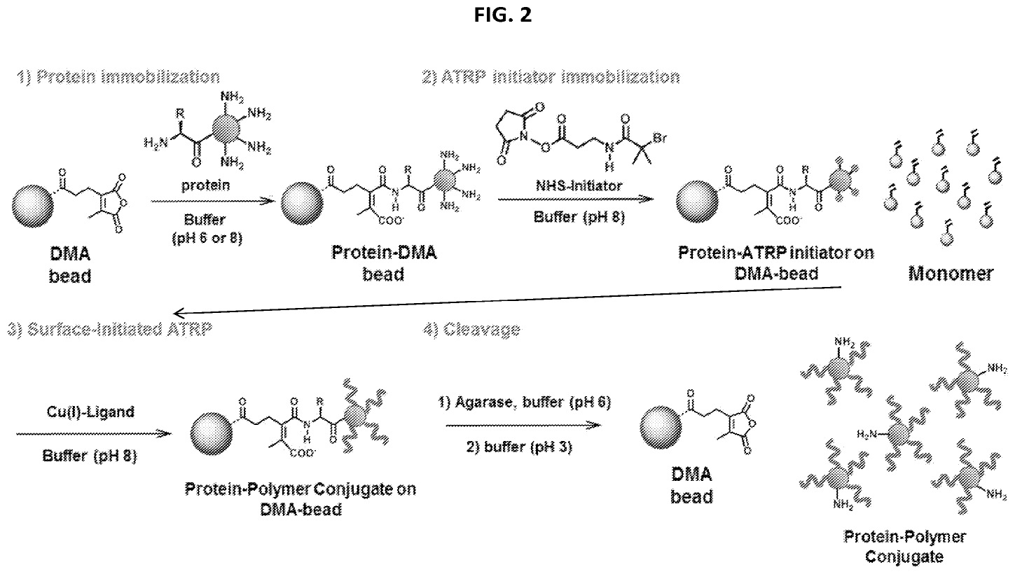

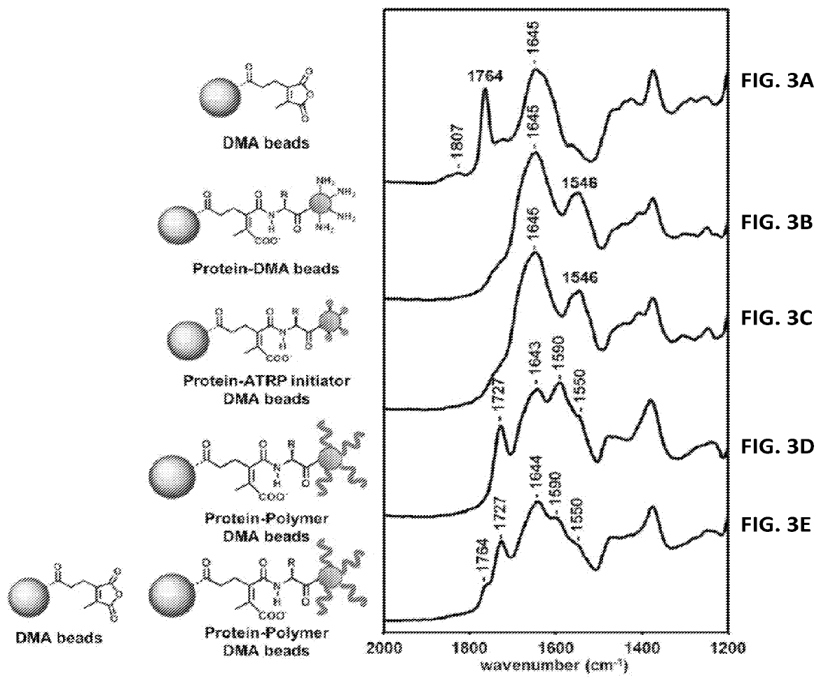

Peptide synthesis from solid supports has traditionally used polystyrene resins (Merrifield, Br. Polym. J. 16:173-178, 1984). Initial experiments, however, demonstrated non-specific hydrophobic adsorption of proteins to dialkyl maleic anhydride modified polystyrene beads. Thus, studies were conducted with hydrophilic supports that might reduce non-specific protein-support binding and ultimately release a grown-from protein-polymer hybrid. Agarose beads are hydrophilic and are stable at extremes of pH, ionic strength, and in the presence of many denaturants. Agarose beads have been widely used in various chromatographic techniques for protein purification (Barbosa et al., Biotechnol. Adv. 33:435-456, 2015). Dialkyl maleic anhydrides can covalently react with primary amines above pH 6, and release below pH 6. Dialkyl maleic anhydride-modified agarose (DMA) beads (45-165 μm) were synthesized (FIG. 1), and the pH-dependence of on and off rates with a cyanine 3 amine fluorescent dye showed that up to 0.25 μmol_{DM}/mL_{beads} would be available for protein attachment during PARIS. Next, each of the four major steps for PARIS (FIG. 2) was performed: protein immobilization onto the DMA-agarose beads at pH 6.0 or pH 8.0; ATRP-initiator immobilization on the agarose-supported protein (using N-2-bromo-2-methylpropanoyl-β-alanine N′-oxysuccinimide bromide (NHS—Br) as described elsewhere; see, Cummings et al., Biomacromolecules 18:576-586, 2017); surface-initiated ATRP to grow polymers from agarose-supported proteins (Murata et al., supra); and cleavage of the resulting protein-polymer conjugates from the agarose supports below pH 6. Each step was characterized with Fourier-transform infrared spectroscopy (FT-IR) (FIGS. 3A-3E).

### Example 3—Protein Reaction with DMA-Agarose

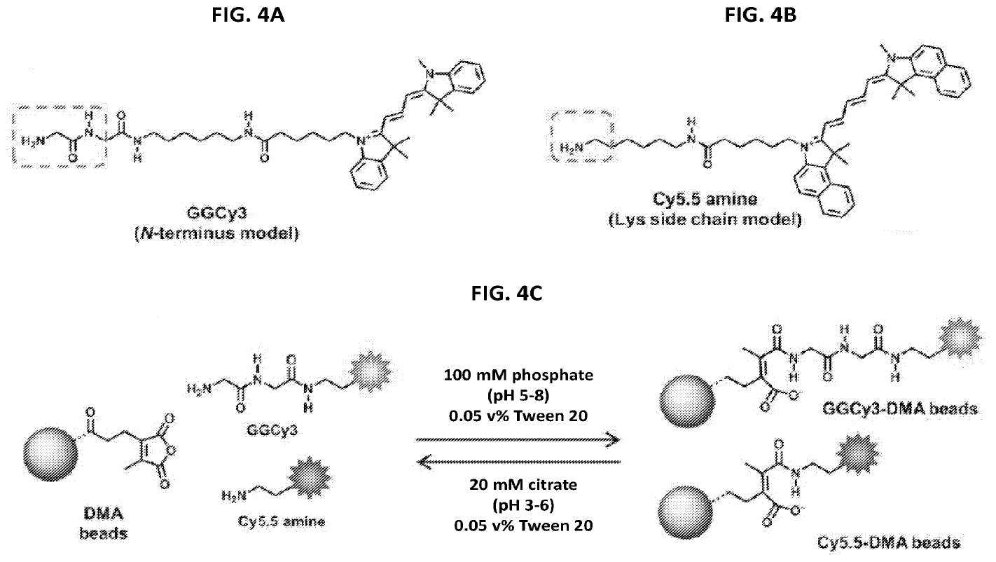

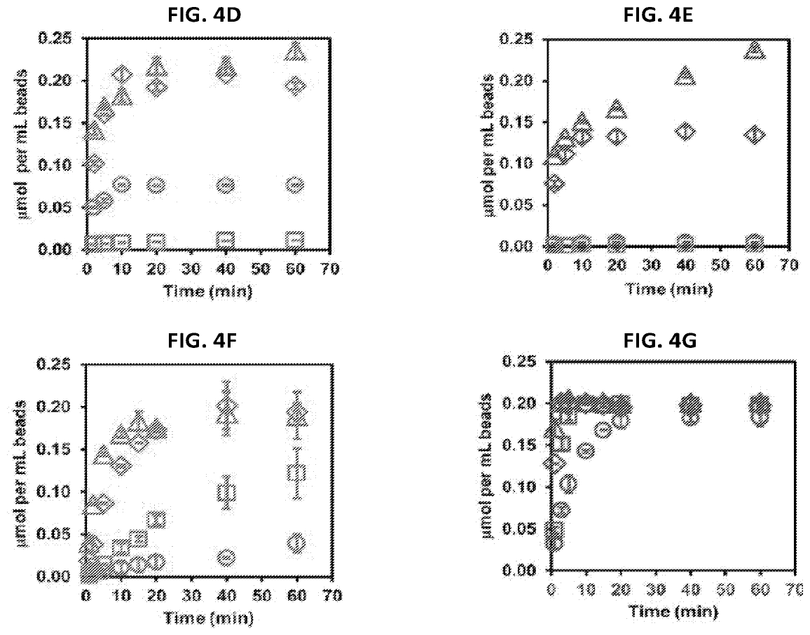

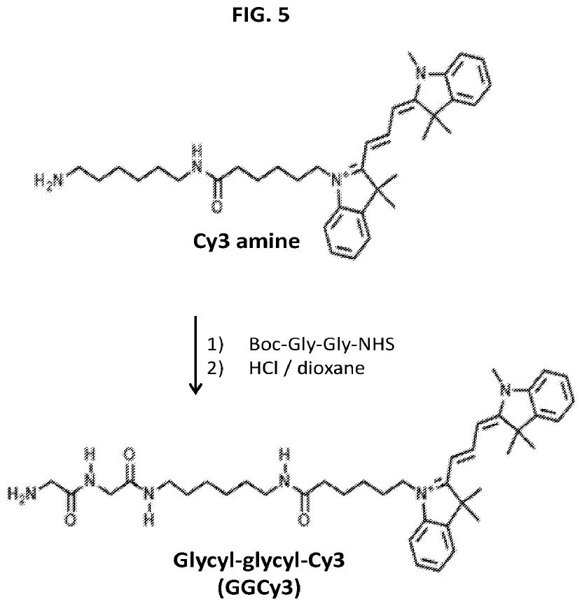

Proteins contain a number of accessible amino groups, including the N-terminal α-amino and lysine side-chain ε-amino groups, that could potentially react with DMA-agarose beads to yield families of immobilized proteins. Studies were conducted to determine whether mostly homogeneous protein-polymer conjugates by preferentially targeting the protein-DMA reaction to the N-terminus of the protein by lowering the reaction pH, since acylation of α-amino groups (N-terminus) typically is preferred at pH 6.5 while ε-amino groups (lysine residues) react efficiently above pH 8.0 (Gaudriault and Vincent, Peptides 13:1187-1192, 1992). Thus, the pH dependence of DMA-lysine and DMA-N-terminal group reactions was first investigated using Cy5.5 amine and glycyl-glycyl-Cy3 (GGCy3) fluorescent dyes (FIGS. 4A-4G and 5-8) as lysine and N-terminal mimics, respectively. The pKa of a lysine side chain is about 10.5-12.0, while the pKa of the N-terminus is about 7.8-8.0. Thus, at a pH below 8.0, the N-terminus will have increased nucleophilicity over lysine residues, potentially leading to preferential immobilization of the α-amino group to the DMA-agarose beads. Binding and release of these model dyes to the beads were determined as a function of pH and time (FIGS. 4D-4G).

Binding studies were performed by incubating the dye with the DMA beads at pH 5-8 over 60 minutes, washing with pH 8 phosphate buffer, releasing the dye at pH 3, and measuring fluorescence in the supernatant at various time points. At pH 7 and 8, both the N-terminal and lysine mimic dyes quickly bound to the DMA beads, with maximum binding occurring within approximately 20 minutes. At pH 5 and 6, however, the binding rate of the N-terminal mimic, GGCy3, was an order of magnitude higher than that for the lysine mimic. Both GGCy3 and Cy5.5 amine showed increased initial binding rates with increased pH. At each pH investigated, the initial binding rate of GGCy3 was higher than that of Cy5.5 amine, indicating that the N-terminus α-amino had a higher binding affinity to the DMA beads. This observation was even more pronounced at pH 5-6. Thus, the data indicated that N-terminal α-amino-targeted protein binding to DMA beads was achievable at pH 6.0, providing evidence for site-specific immobilization.

Release studies also were performed using the GGCy3 and Cy5.5 amine fluorescent model dyes as a function of pH and time. Model dyes that were previously immobilized on the DMA beads at pH 8 were incubated in pH 3 to 6 releasing citrate buffers for 60 minutes. Supernatant fluorescence intensities were measured at various time points. Cy5.5 amine was rapidly released from the DMA beads, with the maximum dye released within about 5 minutes at pH 3, 4, and 6, and within about 20 minutes at pH 5. GGCy3 also showed fast release at pH 3 and 4 within about 20 minutes, but slower release at pH 5 and 6. Interestingly, Cy5.5 amine showed an order of magnitude higher initial release rate than GGCy3 at all pH from 3 to 6. Further, the release rate for both model dyes decreased as pH increased.

Next, the pH dependence of protein immobilization at pH 6.0 and 8.0 and subsequent release over time from pH 3 to 6 was investigated, using chymotrypsin (CT) as a model protein. At pH 6.0, both binding and release occurred at variable rates depending on the reacting amino group. From the model dye data, it was apparent that the binding rate at pH 6.0 was greater than the release rate for the N-terminal mimic, while the opposite was true for the lysine mimic (TABLE 1). The total concentration of bound protein after immobilization (1.82±0.12 and 4.02±0.11 mg CT/mL beads at pH 6.0 and 8.0, respectively) was determined using a bicinchoninic acid (BCA) assay. The concentration of immobilized protein achieved by reacting protein and DMA-agarose beads at pH 6.0 was less than that from the reaction at pH 8.0, again suggesting that this reaction could be site-specific since increased pH would increase the number of ε-amino groups that could react with the DMA, thereby significantly increasing the reaction stoichiometry.

TABLE 1
pH dependence of initial binding and release rates
for GGCy3 and Cy5.5 amine with DMA beads

| | Initial Binding Rate | | | Initial Release Rate | |
|---|---|---|---|---|---|
| | (μmol min⁻¹ · mL⁻¹ beads) | | | (μmol min⁻¹ · mL⁻¹ beads) | |
| pH | GGCy3 | Cy5.5 amine | pH | GGCy3 | Cy5.5 amine |
| 5 | 6.5 ± 0.1 | 0.4 ⊥ 0.1 | 3 | 44.4 ± 1.2 | 170.7 ± 10.1 |
| 6 | 25.3 ± 0.4 | 1.7 ± 0.2 | 4 | 18.8 ± 2.3 | 127.8 ± 5.3 |
| 7 | 51.3 ± 1.4 | 37.9 ± 1.9 | 5 | 2.9 ± 0.2 | 48.5 ± 3.7 |
| 8 | 71.1 ± 0.2 | 54.8 ± 4.2 | 6 | 1.2 ± 0.1 | 32.1 ± 5.7 |

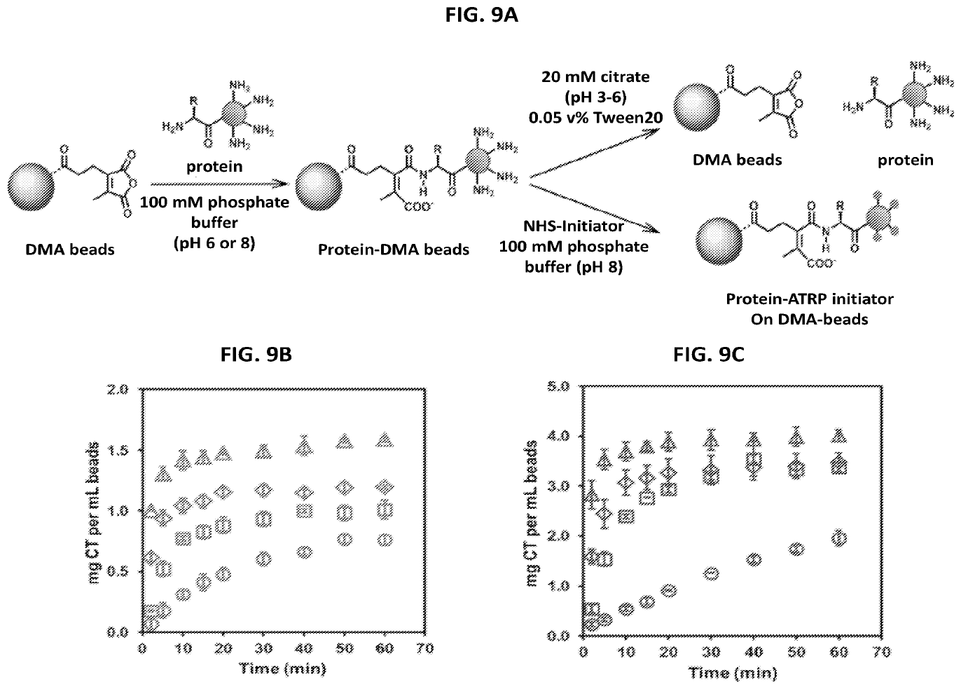

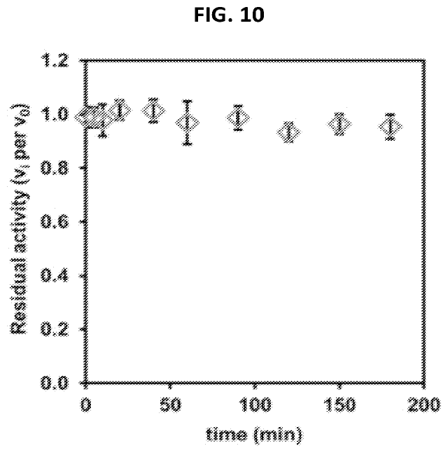

Protein release kinetic studies were then performed over 60 minutes as a function of pH (ranging from pH 3 to pH 6) for proteins initially immobilized at pH 6.0 and 8.0 (FIGS. 9A-9C). In both cases, the total protein recovered and the release rate were increased with decreasing pH, consistent with the model dye experiments. The percent of protein recovered was calculated after 60 minutes for each releasing pH. The highest yields were 88% and 100% at pH 3 for initial immobilizations at pH 6.0 and 8.0, respectively, with the majority of release occurring within the first 5 minutes (TABLE 2). To ensure that CT was not inactivated by incubation in releasing buffer, residual activity was measured over time at pH 3 (FIG. 10). Chymotrypsin was able to recover full activity at neutral pH even after incubation for 3 hours at pH 3. For more sensitive proteins, however, release can be performed at pH 6.0 with 48% recovery. Moreover, in PARIS, the released product (a protein-polymer conjugate) also may have increased acid stability (Murata et al., Biomacromolecules 15:2817-2823, 2014), further protecting the protein from the acidity of releasing buffer. The data presented herein show that protein can be immobilized to and subsequently released from DMA-agarose beads with high recovery and maintained activity. The release is also shown to be pH dependent, albeit with varying degrees, allowing reaction conditions to be customized to match a given protein's sensitivity to acid. Importantly, the most likely application of PARIS will be to perform combinatorial syntheses of protein polymer conjugates for subsequent high throughput screening. In this application, the final yield is less relevant than the ease and speed of synthesis.

TABLE 2
Protein recovered in releasing buffer from pH 3-6

| | Percent recovery from | Percent recovery from |
|---|---|---|
| Releasing pH | pH 6.0 immobilization | pH 8.0 immobilization |
| pH 3 | 87.8 ± 5.9* | 99.8 ± 4.1 |
| pH 4 | 68.8 ± 4.4 | 86.5 ± 5.3 |
| pH 5 | 55.6 ± 5.5 | 84.1 ± 3.1 |
| pH 6 | 41.8 ± 3.5 | 47.8 ± 4.1 |

*Standard deviation from triplicate measurements.

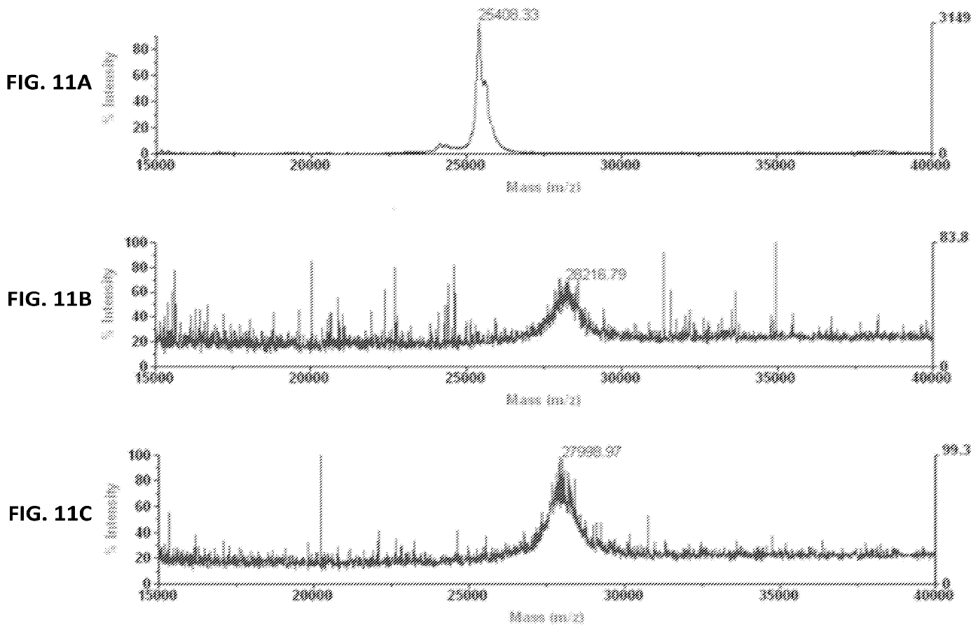

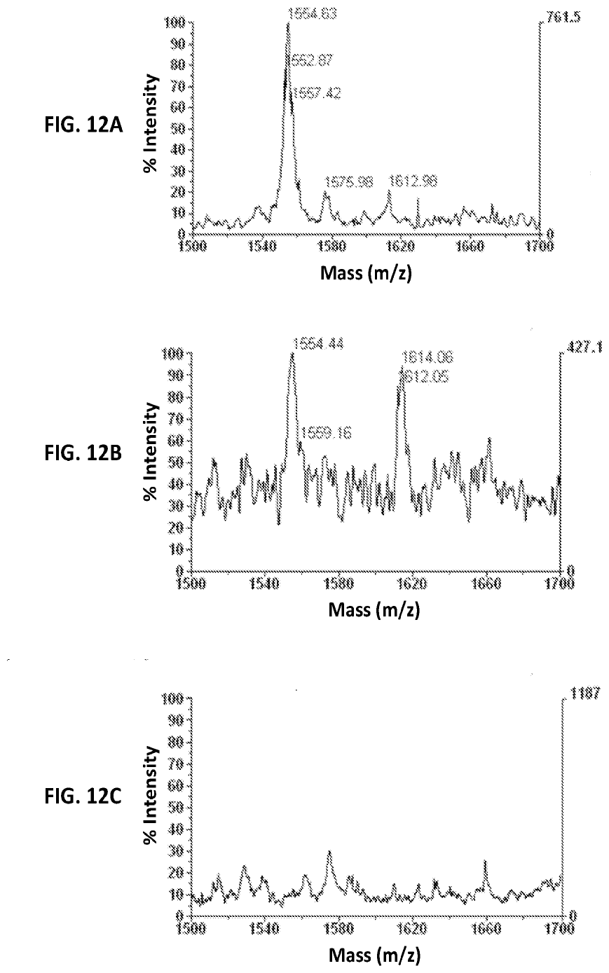

Following immobilization, the next step was ATRP-initiator (NHS—Br) modification of the remaining immobilized accessible protein amino groups, with the assumption that after the protein was immobilized, the remaining amino groups would be available for ATRP-initiator modification. The model dye experiments indicated that the ATRP initiator would not react with the N-terminus since it was already selectively bound to the beads in the case of pH 6.0 immobilization. CT has 15 primary amines: 14 lysine side-chains and an additional α-amino group on the N-terminus. The number of initiator modifications for CT immobilized at pH 6.0 and 8.0 was determined to be 13 and 11, respectively, from matrix-assisted laser desorption/ionization time-of-flight mass spectrometry (MALDI-ToF MS) (FIGS. 11A-11C), as MALDI-ToF MS can be used after trypsin digestion of protein-initiator complexes to determine where initiators had reacted with proteins (Lele et al., Biomacromolecules 6:3380-3387, 2005). The same strategy was used to study modification sites on released initiator-modified CT. No modification of the N-terminal amino group by ATRP-initiator at pH 6.0 was observed, supporting the view that DMA-agarose beads reacted with CT at the N-terminal amino group at low pH (FIGS. 12A and 12B). In contrast, N-terminal modification was observed with ATRP initiator after immobilization at pH 8.0, as indicated by the absence of the peptide fragment peak (FIG. 12C).

### Example 4—PARIS Synthesis of CT-Conjugates

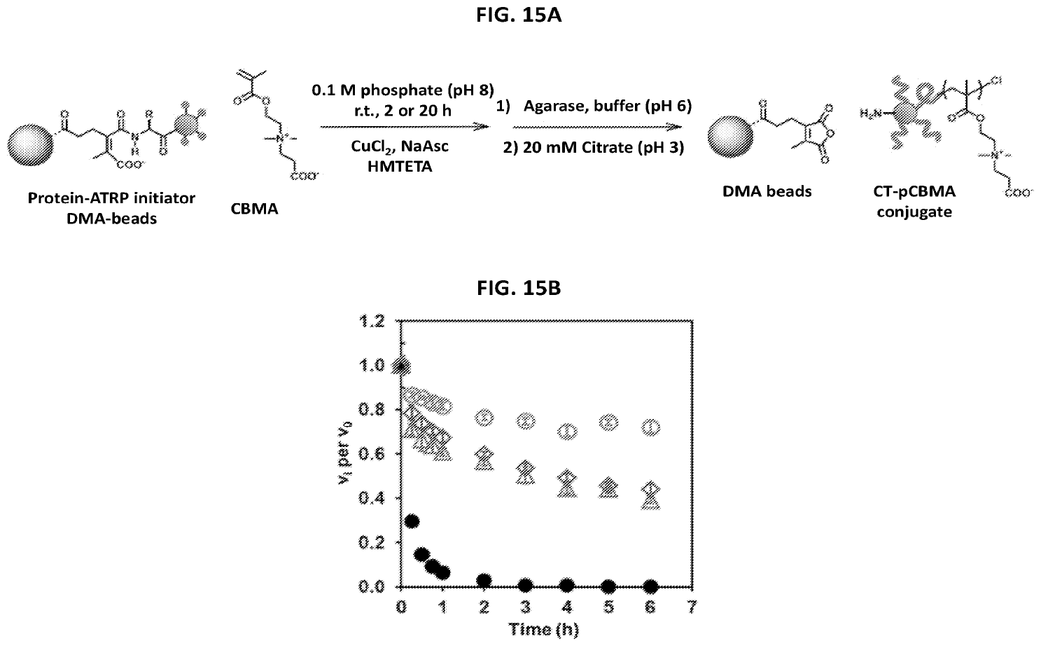

The properties of CT-polymer conjugates have been studied in depth (see, e.g., Gunther et al., Eur. J. Biochem. 267:3496-3501, 2000; Klibanov, Nature 409:241-246, 2001; Falatach et al., Polym. (United Kingdom) 72:382-386, 2015; Sandanaraj et al., J. Am. Chem. Soc. 127:10693-10698, 2005; Hong et al., J. Mol. Catal. B Enzym. 42:99-105, 2006; Kumar and Venkatesu, Chem. Rev. 112:4283-4307, 2012; and Hedstrom, Chem. Rev. 102:4501-4523, 2002). Thus, further experiments were focused on synthesizing and characterizing CT-polymer conjugates grown within, then released from, DMA-agarose beads. Poly(carboxybetaine methacrylate) (pCBMA), a hydrophilic and zwitterionic polymer, was chosen to grow from the surface of the initiated and reversibly immobilized enzyme, since zwitterionic polymers can stabilize CT against irreversible inactivation at extremes of temperatures and pH (Zhang and Cremer, Curr. Opin. Chem. Biol. 10:658-663, 2006; and Cummings et al., Biomacromolecules 18:576-586, 2017). Studies were first aimed at determining whether the pH of immobilization and the agarose beads themselves would impact the structure and function of the subsequently released enzyme. The chemical structure of CT-pCBMA was initially characterized with ¹H NMR and FT-IR (FIGS. 13 and 14), and released PARIS-CT conjugates were further compared to solution-based CT-pCBMA conjugates and native CT (FIGS. 15A and 15B). Following release from the DMA-agarose beads, protein concentrations were determined spectroscopically. Released CT-pCBMA conjugate hydrodynamic diameter (Dₕ) was characterized using dynamic light scattering (DLS). The released conjugates grew in Dₕ from 4.4 nm (native CT) to about 9 nm, independent of immobilization pH. Solution-based CT-pCBMA conjugates were slightly larger than PARIS synthesized conjugates (TABLE 3). The conjugates were further characterized using acid hydrolysis to cleave the polymer, followed by gel permeation chromatography (GPC), which showed polymer molecular weights of 9.2 and 8.2 kDa for CT immobilized at pH 6.0 and 8.0, respectively, with low polydispersity indices (PDI) for each.

TABLE 3
Characterization of released PARIS-synthesized CT conjugates

| | Released | | Cleaved | Estimated | | | |
|---|---|---|---|---|---|---|---|
| CT | CT-pCBMA^{b} | | polymer^{d} | Conjugate | | | |
| immobilization | (mg CT per | Dₕ^{c} | (Mₙ (kDa); | M_{w}^{e} | k_{cat}^{f} | K_{M}^{f} | k_{cat}/K_{M}^{f} |
| (pH) | mL beads) | (nm) | (M_{w}/Mₙ) | (kDa) | (s⁻¹) | (μM) | (s⁻¹ μM⁻¹) |
| 6 | 0.56 ± 0.02 | 9.2 ± 2.4 | 9.2; (1.27) | 115.7 | 33.3 ± 1.0 | 65.5 ± 8.2 | 0.508 ± 0.065 |
| 8 | 1.23 ± 0.01 | 9.1 ± 1.9 | 8.2; (1.26) | 133.1 | 34.6 ± 1.2 | 70.4 ± 10.5 | 0.491 ± 0.075 |
| Native CT | — | 4.4 ± 1.3 | — | — | 34.6 ± 1.4 | 80.3 ± 13.1 | 0.431 ± 0.072 |
| Solution-based | — | 10.9 ± 1.4 | — | — | 21.5 ± 0.8 | 45.0 ± 7.9 | 0.478 ± 0.086 |
| CT-pCBMA^{a} | | | | | | | |

^{a}CT-pCBMA conjugate prepared by solution-based method.
^{b}The concentration of released conjugate based on CT per 1 mL of beads (estimated by UV absorption assay) indicated that there are more possible binding sites at pH 8.0 than at pH 6.0.
^{c}Hydrodynamic diameters (number intensity) of the native CT and CT-pCBMA conjugates, measured using dynamic light scattering in 20 mM sodium citrate (pH 3.0) at 25° C., showed an increase in conjugate size over native CT.
^{d}Number average molar mass of CT-pCBMA conjugates and polydispersity index from GPC.
^{e}Estimated conjugate molecular weight from GPC data.
^{f}Michaelis-Menten kinetic values for CT-catalyzed hydrolysis of suc-AAPF-pNA determined by nonlinear curve-fitting of plots of initial rate versus substrate concentration using Enzfitter software. Conjugates synthesized by PARIS did not alter activity in comparison to solution synthesized CT-pCBMA and native CT.

In further studies, the activity of released PARIS-CT conjugates was compared to that of native CT and CT-pCBMA grown in solution. The turnover number and Michaelis constant (k_{cat} and K_{M}) showed that all conjugates had activities similar to that of N-succinyl-L-Ala-L-Ala-L-Pro-L-Phe-p-nitroanilide (suc-AAPF-pNA) (TABLE 3). Since the PARIS conjugates immobilized at pH 6.0 and 8.0 had similar activities, the location of protein-bead immobilization did not significantly alter CT activity. Naturally, if polymer growth from the terminal amino group was performed on a protein that was sensitive to N-terminal modification, the activity of the conjugate would likely be lower for PARIS conjugates immobilized at pH 6.0. It also is worth noting that protein-polymer conjugates typically have reduced catalytic efficiencies through a combined decrease in k_{cat} due to structural stiffening (Rodriguez-Martinez et al., Biotechnol. Bioeng. 101:1142-1149, 2008) and decrease in K_{M} due to the polymer's superhydrophilicity (Cummings et al., Biomacromolecules 18:576-586, 2017). The overall catalytic productivity, k_{cat}/K_{M}, of PARIS CT-pCBMA was similar to both solution-synthesized CT-pCBMA and native CT (TABLE 3).

Next, studies were conducted to demonstrate that PARIS conjugates maintained the same stabilizing effect as conventionally synthesized protein-polymer conjugates in solution. Protein-polymer conjugates synthesized in solution can have enhanced stability to extremes of pH (Thomas et al., Nature 318:375-376, 1985), temperatures (Cummings et al., Biomacromolecules 15:763-771, 2014), and organic solvents (Cummings et al., ACS Macro Lett. 5:493-497, 2016) due to the protective polymer coating. PAR.IS-CT conjugates significantly enhanced the thermostability of the enzyme at 50° C. as compared to native CT; as shown in FIG. 15B, native CT was irreversibly inactivated after about 2 hours at 50° C., but both PARIS CT-pCBMA and solution-based CT-pCBMA had significantly increased stability. Interestingly, PARIS conjugates that were initially immobilized at pH 6.0 were significantly more stable after 6 hours at 50° C. than those immobilized at pH 8.0. Proteins can include regions that are particularly susceptible to stability-impacting modifications, and in many proteins, the N-terminus is the most thermally sensitive region (see, e.g., Chatterjee and Sengupta, Mol. Biosyst. 12:1139-1150, 2016; Jiang et al., Phys. Chem. Chem. Phys. 18:21340-21350, 2016; Wang et al., Sci. Rep. 4:7284, 2014; and Šanderová et al., Biochim. Biophys. Acta—Proteins Proteomics 1804:147-155, 2010). Additionally, enzymes with increased thermostability often are more hydrophobic (Ward and Moo-Young, Biotechnol. Adv. 6:39-69, 1988; and Vieille and Zeikus, Trends Biotechnol. 14:183-190, 1996). Since the N-terminus of CT is located in a largely hydrophobic area, disruption of this hydrophobicity by initiator modification and consequent hydrophilic polymer growth could have decreased the thermostability of the pH 8.0 PARIS CT-pCBMA.

### Example 5—ATRP Polymerization Kinetics

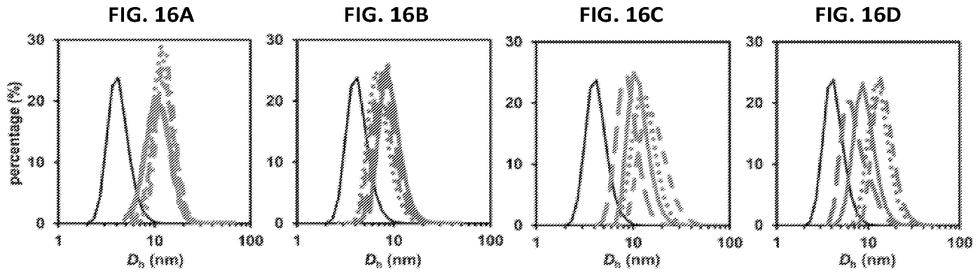

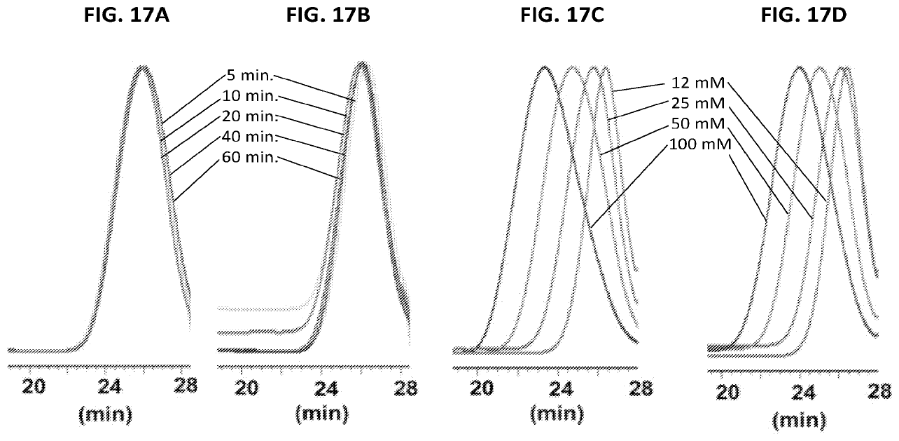

To explore whether the DMA-agarose beads used in PARIS disrupted ATRP reaction kinetics, ATRP kinetics of PARIS and solution-synthesized conjugates were compared (TABLES 4 and 5). Polymer growth was monitored over 60 minutes for a fixed monomer concentration of 25 mM by measuring Dₕ of conjugates at specified time points using dynamic light scattering (TABLE 4 and FIGS. 16A-16D). Additionally, polymers were released from the conjugates at each specified time point, and their molecular weights and polydispersity indices (PDIs) were measured using gel permeation chromatography (FIGS. 17A-17D). Polymer molecular weight did not increase significantly after 5 minutes, indicating that the ATRP reaction was rapid and was completed within the first 5 minutes. Comparison of PARIS and solution-based conjugates showed similar overall polymer growth, but PARIS conjugates displayed lower PDIs. The agarose bead pore size used in PARIS was about 30 nm, which could have provided a uniform microenvironment during chain propagation, leading to lower PDIs and more uniform conjugates. In conventional solution-based ATRP, polymer molecular weight can be tuned to a desired value by increasing the monomer to initiator ratio during the ATRP reaction. To show that conjugate size could be easily varied by PARIS, the monomer concentration was systematically increased from 12.5 to 100 mM, and conjugate Dₕ, polymer molecular weights, and PDIs were determined for a 60 minute reaction time (TABLE 5). As expected, conjugate Dₕ and polymer molecular weight increased with increasing monomer concentration for both PARIS and solution-based conjugate synthesis (each maintaining low PDIs; FIGS. 16A-16D and 17A-17D). In summary, these exciting results demonstrated that for pCBMA, highly uniform conjugates were synthesized by PARIS within 5 minutes, polymer molecular weights were predictably controlled, and PARIS conjugates were similar to, and perhaps even more homogeneous than, solution-synthesized conjugates.

TABLE 4
PARIS and solution-based conjugate ATRP kinetics

| | | | Dₕ^{b} | Cleaved pCBMA^{c} | | |
|---|---|---|---|---|---|---|
| Sample^{a} | | | (nm) | Mₙ | M_{w} | PDI |
| Solution | 5 | min | 12.3 ± 4.2 | 10100 | 14400 | 1.42 |
| | 10 | min | 13.5 ± 3.9 | 9800 | 13900 | 1.44 |
| | 20 | min | 12.8 ± 7.5 | 10000 | 14700 | 1.47 |
| | 40 | min | 11.1 ± 7.3 | 9800 | 14200 | 1.45 |
| | 60 | min | 11.3 ± 7.8 | 11000 | 16700 | 1.50 |
| PARIS | 5 | min | 7.3 ± 1.2 | 9600 | 12300 | 1.28 |
| | 10 | min | 7.9 ± 2.5 | 9900 | 12800 | 1.29 |
| | 20 | min | 9.3 ± 1.3 | 9600 | 12500 | 1.30 |
| | 40 | min | 8.6 ± 2.0 | 10000 | 13300 | 1.33 |
| | 60 | min | 9.4 ± 1.4 | 10600 | 14700 | 1.39 |

TABLE 5
PARIS and solution-based conjugate ATRP kinetics

| | |
|---|---|
| Dₕ^{b} | Cleaved pCBMA^{c} |

| | | | | |
|---|---|---|---|---|
| Sample^{a} | (nm) | Mₙ | M_{w} | PDI |

Solution

| | | | | | |
|---|---|---|---|---|---|
| 12.5 | mM | 9.0 ± 2.9 | 8200 | 9900 | 1.18 |
| 25 | mM | 11.8 ± 2.4 | 11100 | 15100 | 1.35 |
| 50 | mM | 13.6 ± 4.3 | 16100 | 25600 | 1.61 |
| 100 | mM | 17.0 ± 8.4 | 27900 | 48800 | 1.77 |

PARIS

| | | | | | |
|---|---|---|---|---|---|
| 12.5 | mM | 7.4 ± 3.9 | 7000 | 7700 | 1.10 |
| 25 | mM | 9.4 ± 2.5 | 9600 | 12500 | 1.30 |
| 50 | mM | 13.5 ± 3.2 | 15200 | 21500 | 1.43 |
| 100 | mM | 15.2 ± 2.3 | 23400 | 36000 | 1.53 |

### Example 6—PARIS Synthesis of Protein-Conjugates

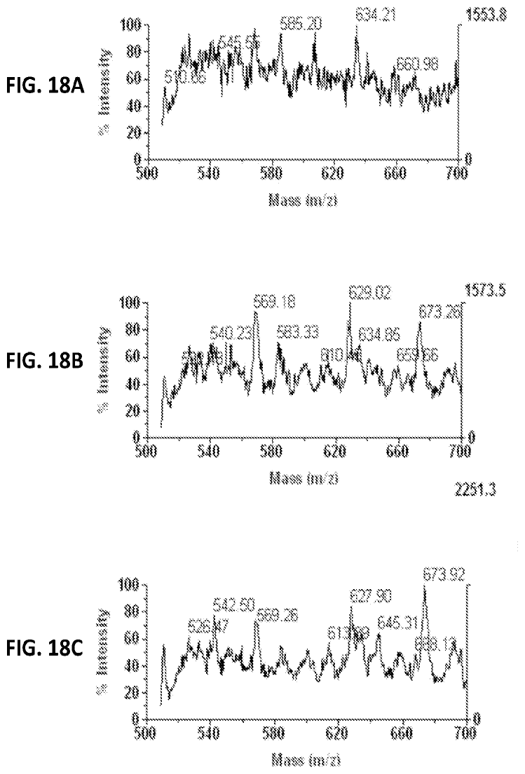

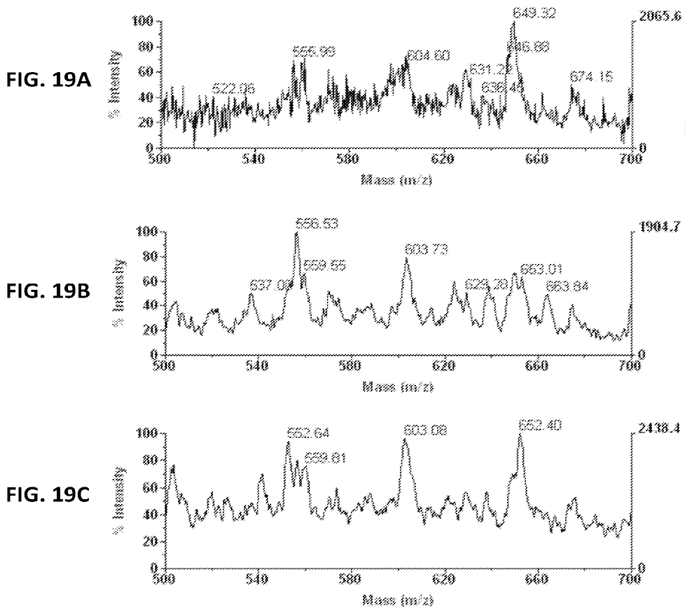

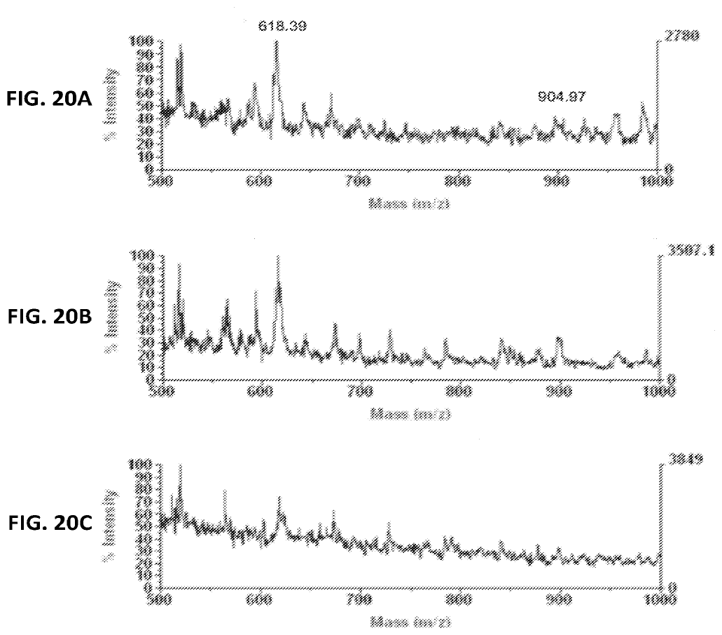

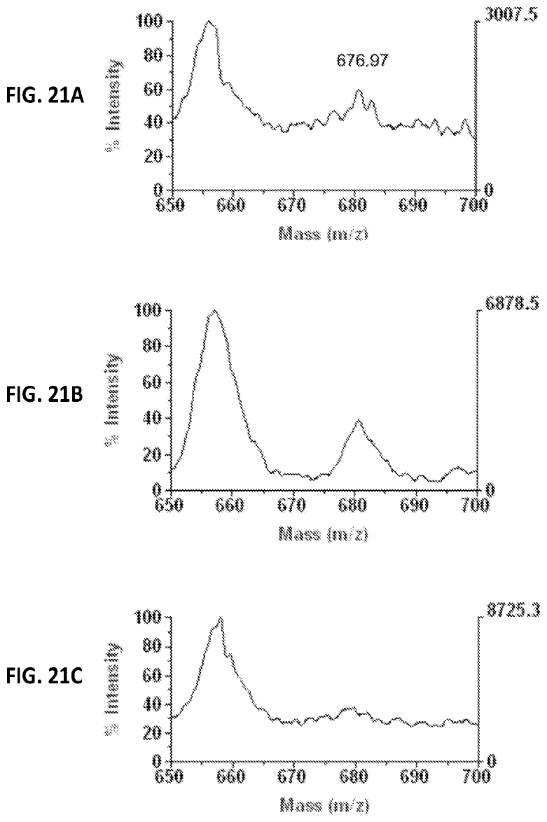

Although the results with CT-conjugates synthesized by PARIS were exciting, an important step was to demonstrate that PARIS can be applied to a breadth of unrelated proteins. To demonstrate that solution synthesized conjugates were nearly identical to PARIS-synthesized conjugates, a series of proteins was selected with varying sizes, structures, and N-terminus accessibility: lysozyme (Lyz, M_{w,monomer}=14.3 kDa), avidin (M_{w,tetramer}=68 kDa), acetylcholinesterase (AChE, M_{w,tetramer}=272 kDa), and uricase (Uox, M_{w,tetramer}=140 kDa). As with CT, it was first determined whether the reaction between DMA-agarose and protein was N-terminus specific for each protein. Tryptic digestion studies of the acetylcholinesterase-initiator complex showed that, as observed with CT, the peptide fragment containing the N-terminus (904.97 m/z, [M+H]+) was absent when AChE was immobilized at pH 8.0 (FIGS. 20A-20C and TABLE 6). Analogous results also were obtained for the uricase-initiator complex. The peptide fragment containing the N-terminus was present in both native uricase and the uricase-initiator complex from the pH 6.0 experiment (676.97 m/z, [M+H]+), but absent when the uricase-initiator was prepared after immobilization at pH 8.0 (FIGS. 21A-21C and TABLE 6). Avidin and lysozyme have N-termini that are inaccessible, so it was anticipated that they would not have site-specific reactions. Indeed, native avidin and avidin-initiator complex that had been reacted with DMA-agarose at both pH 6.0 or pH 8.0 retained the peptide fragment containing the N-terminus (555.99 m/z, [2M+ACN+Na]⁺), indicating that the N-terminus was not modified with the NHS—Br initiator under any of the reaction conditions (FIGS. 19A-19C and TABLE 6). For lysozyme, since the N-terminus was a lysine residue, 2-nitro-5-thiocyanatobenzoic (NTCB) acid digestion studies were conducted, in which NTCB reacted specifically with cysteine thiols by cyanylation. Similarly to avidin, the N-terminus peptide fragment (585.20 m/z, [M+ACN+Na]+) in lysozyme was still free after immobilization at pH 6.0 and 8.0 (FIGS. 18A-18C and TABLE 6). Thus, the data from all proteins showed that the immobilization reaction was N-terminus selective at pH 6.0, as long as the α-amino group was surface accessible, and was independent of protein size and quaternary structure.

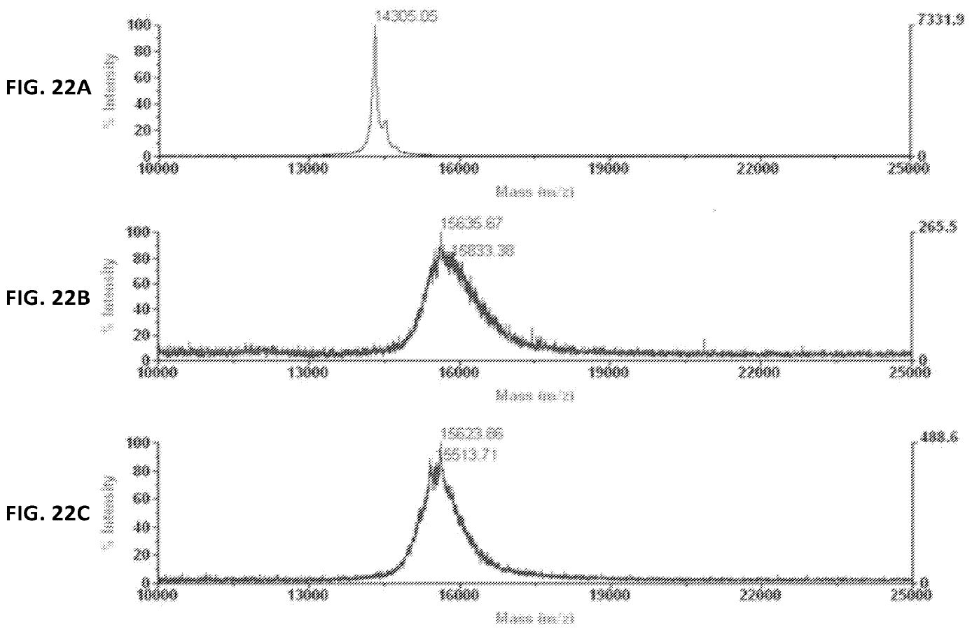

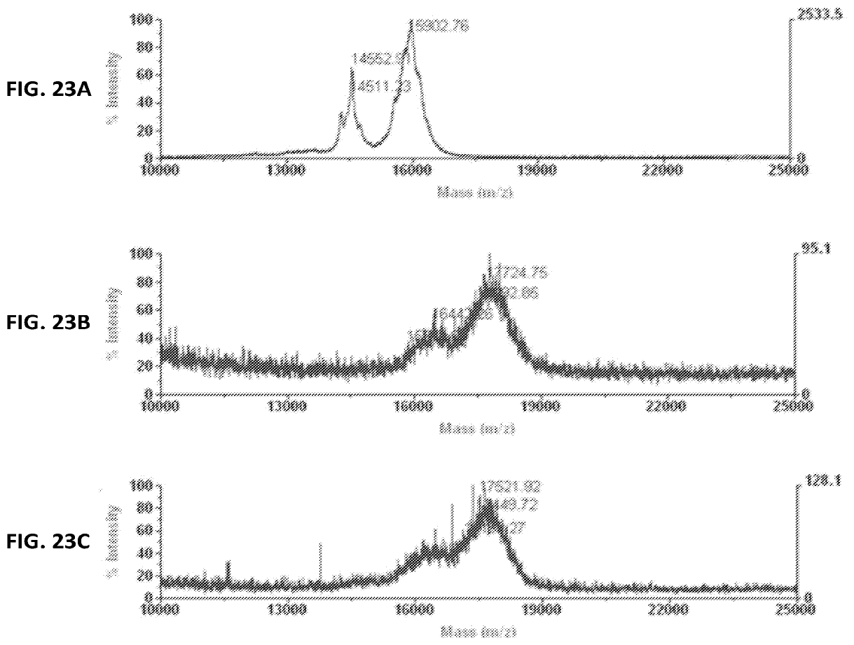

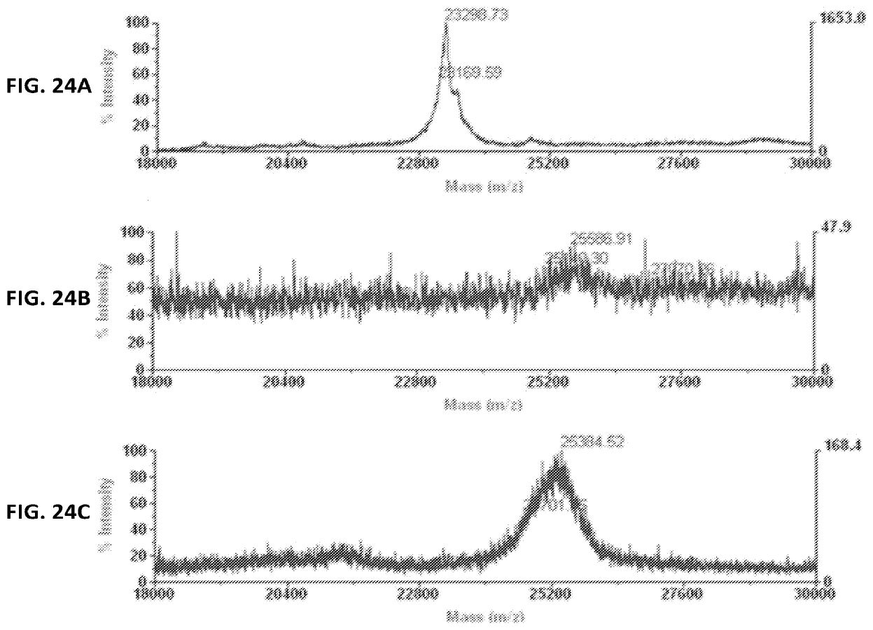

Initiator-modified proteins also were characterized by MALDI-ToF MS to determine the number of modification sites after initial immobilization at pH 6.0 and 8.0 (FIGS. 22A-22C, 23A-23C, and 24A-24C). After successful protein-ATRP with pCBMA, followed by release, the protein-polymer conjugates were fully characterized (TABLE 7). The percent of recovered conjugate was decreased in comparison to the prior released native protein experiments, with the highest conjugate recovery obtained for the smallest starting protein size (51% for lysozyme). This result was not necessarily surprising, since larger conjugates could become more trapped inside the pores after polymer growth, thus hindering full release. To increase release, agarose bead pore size can be increased to accommodate larger proteins, multiple incubation steps in releasing buffer can be performed in series, and/or the ratio of releasing buffer to agarose bead solution can be increased; results for agarase-induced release optimization are shown in FIG. 25.

TABLE 6
Peak analysis of peptide fragments
after trypsin digestion or NTCB digestion

| | | Expected | Observed |
|---|---|---|---|
| | Peptide | mass | mass |
| Protein | Fragment | (m/z) | (m/z) |
| Chymotrypsin | ¹CGVPAIQPVLSGLSR | 1554.78 | 1554.63 |
| | (SEQ ID NO: 1) | [M + H]⁺ | |
| Acetylcholine- | ¹SELLVNTK | 904.05 | 904.97 |
| sterase | (SEQ ID NO: 2) | [M + H]⁺ | |
| Uricase | ¹MAHYR | 677.79 | 676.97 |
| | (SEQ ID NO: 3) | [M + H]⁺ | |
| Avidin | ¹AR | 554.58 | 555.99 |
| | | [2M + ACN + | |
| | | Na]⁺ | |
| Lysozyme | ¹KVFGR | 586.60 | 585.20 |
| | (SEQ ID NO: 4) | [M + ACN + | |
| | | Na]⁺ | |

TABLE 7
PARIS synthesis and characterization of ranging proteins

| | | Protein | Released | | Cleaved |
|---|---|---|---|---|---|
| | | on beads^{a} | Protein-pCBMA^{b} | | Polymer^{d} |
| | Ratio of | (mg | (mg protein per | Dₕ^{c} | Mₙ (kDa) |
| | activity^{e} | protein per | mL beads) | (nm) | (M_{w}/Mₙ) |
| Protein | (PARIS:solution) | mL beads) | (% recovery) | (solution) | (solution) |
| Lysozyme | 1.94 ± 0.13 | 1.89 | 0.97 (51%) | 9.4 ± 1.8 | 19.8 (1.37) |
| | | | | (7.1 ± 2.6) | (15.0 (1.26)) |
| Avidin | 1.01 ± 0.14 | 3.54 | 1.05 (30%) | 13.2 ± 2.8 | 13.2 (1.30) |
| | | | | (21.7 ± 6.4) | (16.1 (1.34)) |
| Chymotrypsin | 1.03 ± 0.24 | 2.01 | 0.89 (44%) | 8.1 ± 0.7 | 10.6 (1.39) |
| | | | | (10.9 ± 1.4) | (11.0 (1.50)) |
| Acetyl- | 1.09 ± 0.08 | 0.79 | 0.16 (20%) | 13.0 ± 2.0 | 14.5 (1.35) |
| cholinesterase | | | | (13.9 ± 5.2) | (7.2 (1.33)) |
| Uricase | 2.33 ± 0.15 | 0.43 | 0.09 (21%) | 10.9 ± 1.7 | 32.4 (1.50) |
| | | | | (12.5 ± 5.0) | (14.7 (1.30)) |

^{a}Concentration of immobilized protein per 1 mL of beads determined by microBCA protein assay.
^{b}Concentration of released conjugate per 1 mL of beads and percentage of recovered protein determined by microBCA protein assay.
^{c}Hydrodynamic diameter measured by dynamic light scattering (number distribution).
^{d}Number average molecular weight and polydispersity index of cleaved pCBMA from PARIS conjugates determined by gel permeation chromatography and compared to solution-based conjugates.
^{e}Ratio of conjugate activity of PARIS to solution-based approaches. Error bars represent standard deviation from triplicate measurements.

Hydrodynamic diameters also were similar for each PARIS- and solution-synthesized conjugate pair. To provide complete characterization, polymers were released and analyzed using GPC. All polymers maintained low PDIs, whether synthesized by PARIS or in solution.

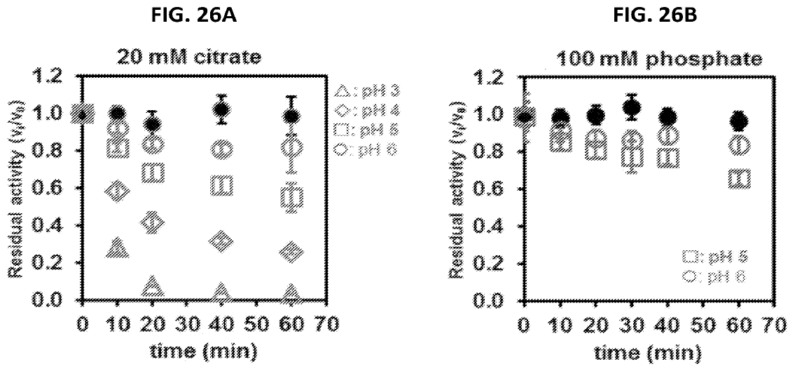

Studies also were conducted to determine whether the PARIS-synthesized conjugates had the same activity as solution-synthesized conjugates. Activity assays specific for each enzyme were performed (TABLES 8-11), and activities were reported as a ratio of PARIS- to solution-synthesized conjugates (TABLE 7e). In all cases, PARIS conjugates had maintained activities compared to solution-based conjugates. Additionally, lysozyme and uricase conjugates synthesized by PARIS had twice the activity of their solution-based counterparts. Thus, PARIS may generate more active conjugates than solution-based methods. The acetylcholinesterase enzyme was sensitive to release at pH 3, but successful release at pH 5 enabled comparison with a solution-synthesized conjugate (FIGS. 26A and 26B). Overall, these studies demonstrated that PARIS is a reliable, straightforward protein-polymer conjugate synthesis method, that the PARIS data were comparable to conventional solution-generated data in terms of both physical and functional bioconjugate properties, and that conjugate synthesis via PARIS chemistry was not only suitable for a breadth of proteins, but also easily tunable for a specific protein's sensitivity. It is to be noted that the PARIS strategy is not limited to the release chemistry used for the exemplary solid-state protein-ATRP studies described herein.

TABLE 8
Michaelis-Menten kinetic values for AChE-catalyzed
hydrolysis of acetylthiocholine iodide

| | kcat | KM | kcat/KM | Ratio of enzymatic efficiency |
|---|---|---|---|---|
| AChE-pCBMA | (s⁻¹) | (mM) | (mM⁻¹ s⁻¹) | (PARIS:solution) |
| PARIS | 0.89 ± 0.02 | 0.179 ± 0.010 | 4.97 ± 0.30 | 1.09 ± 0.08 |
| Solution | 1.30 ± 0.02 | 0.285 ± 0.011 | 4.56 ± 0.19 | |

TABLE 9
Enzymatic activity of Lyz-pCBMA

| | M. lysodeikticus | | N-acetyl β-chitotriose | |
|---|---|---|---|---|
| | ΔA450 min⁻¹ | Ratio of lysis | ΔA405 min⁻¹ | Ratio of hydrolysis |
| | (×10⁻⁵) | (PARIS:solution) | (×10⁻⁵) | (PARIS:solution) |
| PARIS | 91.9 ± 0.2 | 1.94 ± 0.13 | 3.60 ± 0.65 | 1.95 ± 0.53 |
| Solution | 47.3 ± 1.9 | | 1.85 ± 0.56 | |

TABLE 10
Binding affinity of HABA to Avi-pCBMA conjugates

| | | | Ratio of binding |
|---|---|---|---|
| | | Bound HABA | affinity of HABA |
| Avi-pCBMA | ΔA500 | (μM) | (PARIS:solution) |
| PARIS | 0.0316 ± 0.003 | 0.93 ± 0.13 | 1.01 ± 0.14 |
| Solution | 0.0313 ± 0.001 | 0.92 ± 0.04 | |

[Avi]₀ = 2 μM, ε₅₀₀ = 34000 M⁻¹

TABLE 11
Enzymatic activity of Uox-pCBMA conjugates

| | Δ A290 min⁻¹ | | Ratio of activity |
|---|---|---|---|
| Uox-pCBMA | (×10⁻⁵) | U g⁻¹ | (PARIS:solution) |
| PARIS | 17.2 ± 1.10 | 0.70 ± 0.05 | 2.33 ± 0.15 |
| Solution | 7.45 ± 2.75 | 0.30 ± 0.11 | |

ε₂₉₀ = 12300 M⁻¹

### Example 7—Facile Automated One-Pot PARIS

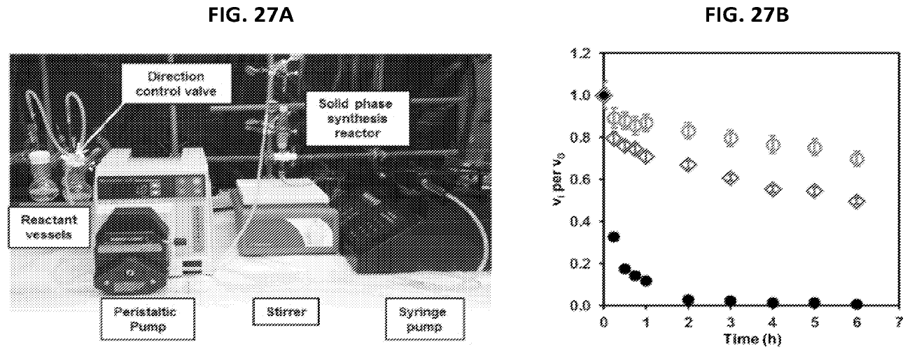

The PARIS method can be used to generate protein polymer conjugates in hours rather than weeks, using simplified chemistry that can be partially or fully automated. A PARIS-flow reactor was developed in order to provide a system for flowing reactants into a column reactor, removing unreacted initiator, removing unreacted monomers, and purifying the conjugates by release from the beads (FIG. 27A). Using this system, CT was bound to the DMA-agarose beads, reacted with initiator, and then released from the DMA-beads in the reactor after polymerization. PARIS synthesis of CT-pCBMA in the flow reactor was compared to batch synthesis, revealing that conjugates synthesized in batch- and flow-modes were structurally and functionally similar. The Dₕ of the conjugates released from the automated flow-based reactor were 8.7 and 9.2 nm for CT initially immobilized at pH 6.0 and 8.0, respectively (TABLE 12). Similarly (and in agreement with results from the batch synthesis), both PARIS conjugates synthesized in the flow reactor had enhanced thermostability at 50° C. relative to native CT, while the most thermostable conjugate was CT-pCBMA that had been immobilized at pH 6.0 (FIG. 27B).

Conventional protein-polymer conjugates that are grown from proteins require initiator modification, followed by days of separation and purification of initiator-modified proteins from excess initiator, and then 2-4 days of polymerization and final purifications. The flow reactor experiment described above was completed in less than 6 hours. While the individual steps of conjugate synthesis between solution-based and PARIS-based approaches were similar, the PARIS purification steps were rapid and readily automated. This reduction in synthesis and purification time, coupled with the ability to multiplex the system, removed the complexity barrier from protein-polymer conjugate synthesis. Since PARIS conjugates were similar in structure and function to solution-based conjugates, an attractive feature of the flow reactor design was that high throughput synthesis would allow more rapid screening of a multitude of protein-polymer conjugates.

Thus, PARIS is a synthetic approach that allows solid-phase synthesis of grafted-from protein-polymer conjugates. PARIS generates conjugates with similar structures and functions to traditional protein-ATRP in solution. Importantly, PARIS can be performed in a simple flow reactor, opening the door to automated and combinatorial protein polymer conjugate syntheses.

TABLE 12
Characterization of CT conjugates by PARIS flow reactor

| | | | | Released | Released | |
|---|---|---|---|---|---|---|
| | Protein on | Released | Number of | CT-pCBMA | CT-pCBMA | |
| CT | beads^{a} | CT-initiator^{b} | initiators on | by UV abs ^{d} | by activity^{e} | |
| Immobilization | (mg CT per | (mg CT per | the | (mg CT per | (mg CT per | Dₕ^{f} |
| (pH) | mL beads) | mL beads) | conjugate^{c} | mL beads) | mL beads) | (nm) |
| 6 | 1.1 | 0.6 | 7.2 | 0.37 | 0.13 | 8.7 ± 0.1 |
| 8 | 2.2 | 1.7 | 10.4 | 1.53 | 0.96 | 9.2 ± 0.4 |

^{a}Concentration of immobilized CT determined by a microBCA protein assay. The concentration of CT on the beads at pH 6.0 is less than pH 8.0, indicating more possible binding sites at pH 8.0.
^{b}Concentration of released CT-initiator determined by a microBCA protein assay.
^{c}Average number of initiators per CT determined by fluorescamine amine assays using standard protocols.
^{d} Concentration of released conjugates estimated by UV absorption.
^{e}Concentration of released conjugates estimated by enzyme activity using suc-AAPF-pNA as a substrate.
^{f}Hydrodynamic diameter of the CT-pCBMA conjugates was measured using dynamic light scattering in 20 mM sodium citrate (pH 3.0) at 25° C., showing an increase in conjugate size over native CT (Dₕ = 4.4 nm, number distribution).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for generating a protein-polymer conjugate, comprising:
attaching a protein of interest to a solid support adapted for coupling to said protein of interest;
contacting said protein of interest with an initiator; and
coupling a first monomer subunit of a polymer to said protein of interest and, optionally, coupling further monomer subunits to said protein via said first monomer subunit, thus generating said protein-polymer conjugate.

2. The method of claim 1, wherein said solid support has been modified for reversible coupling to said protein of interest, such that said method comprises reversibly coupling said protein of interest to said solid support.

3. The method of claim 2, further comprising releasing said protein-polymer conjugate from said solid support.

4. The method of claim 1, comprising non-reversibly attaching said protein of interest to said solid support.

5. The method of claim 1, wherein said protein of interest is an enzyme, a hormone, a cytokine, an antibody, an antigen, an or anti-coagulation protein.

6. The method of claim 5, wherein said protein of interest is an enzyme selected from the group consisting of a protease, an esterase, a dehydrogenase, a hydrolase, and a kinase.

7. The method of claim 1, wherein said solid support is hydrophilic.

8. The method of claim 1, wherein said solid support comprises a polystyrene resin, agarose beads, cross-linked polyethylene glycol (PEG) beads, or magnetic (nano)-beads.

9. The method of claim 1, wherein the solid support comprises agarose beads.

10. The method of claim 1, wherein said solid support comprises a functional group comprising a maleic anhydride, and wherein said method comprises attaching said protein of interest to said solid support via reaction of said maleic anhydride with an amino group on said protein of interest.

11. The method of claim 10, wherein said maleic anhydride is dimethyl maleic anhydride (DMA).

12. The method of claim 10, comprising attaching said protein of interest to said solid support at a pH between 5.5 and 6.5, such that said protein of interest is attached to the solid support via a covalent bond formed between said maleic anhydride and the N-terminal amino group of said protein.

13. The method of claim 10, further comprising exposing the protein-polymer conjugate on said solid support to a pH between about 3 and about 4, such that said protein-polymer conjugate is released from said solid support.

14. The method of claim 13, further comprising, prior to exposing the protein-polymer conjugate on said solid support to a pH between about 3 and about 4, contacting said protein-polymer conjugate on said solid support with agarase.

15. The method of claim 1, wherein said solid support comprises a functional group comprising a disulfide bond, and wherein said method comprises attaching said protein of interest to said solid support via reaction of said disulfide bond with a thiol group of said protein of interest.

16. The method of claim 15, further comprising contacting said protein-polymer conjugate on said solid support with a reducing agent, such that said protein-polymer conjugate is released from said solid support.

17. The method of claim 16, wherein the reducing agent comprises dithiothreitol.

18. The method of claim 1, wherein said solid support comprises a ligand with affinity for said protein of interest, and wherein said method comprises attaching said protein of interest to said solid support via interaction with said ligand.

19. The method of claim 18, wherein said ligand is a substrate for said protein of interest.

20. The method of claim 18, wherein said ligand is an inhibitor of said protein of interest.

21. The method of claim 1, wherein said solid support comprises a nickel-imidodiacetic acid (Ni-IDA) resin, and wherein said method comprises attaching said protein of interest to said solid support via interaction of said Ni-IDA resin with a histidine residue of said protein.

22. The method of claim 21, further comprising exposing said protein-polymer conjugate on said solid support to a pH of about 2.5, such that said protein-polymer conjugate is released from said solid support.

23. The method of claim 21, further comprising contacting said protein-polymer conjugate on said solid support with imidazole or ethylenediamenetetraacetic acid (EDTA), such that said protein-polymer conjugate is released from said solid support.

24. The method of claim 1, wherein said initiator is an atom transfer radical polymerization (ATRP) initiator.

25. The method of claim 24, wherein said ATRP initiator is N-2-bromo-2-methylpropanoyl-γ-alanine N′-oxysuccinimide bromide (NETS-Br).

26. The method of claim 1, wherein said protein of interest is an enzyme, and wherein said method further comprises, prior to or simultaneously with contacting said protein of interest with said initiator, said polymer subunit, or both, contacting said protein of interest with a substrate for said enzyme.

27. The method of claim 1, wherein said polymer is a zwitterionic polymer.

28. The method of claim 1, wherein said polymer comprises a methacrylate, acrylate, acrylamide, styrenic, or acrylamide-styrenic or a combination thereof.

29. The method of claim 1, wherein said polymer is poly(carboxybetaine methacrylate) (pCBMA).

30. The method of claim 1, further comprising at least one washing step selected from the group consisting of:
washing uncoupled protein of interest away from said solid support prior to adding said initiator,
washing excess initiator away from said protein of interest on said solid support prior to coupling said first polymer subunit to said protein of interest, and washing uncoupled polymer subunits away from said protein-polymer conjugate on said solid support.

31. The method of claim 1, wherein said attaching, contacting, and coupling steps are conducted in an automated system.

32. The method of claim 31, wherein said automated system is a flow through system.

33. A method for generating a protein-polymer conjugate, comprising:
coupling a protein of interest to a solid support, wherein said solid support comprises DMA-modified agarose beads, and wherein said coupling results in covalent attachment of said protein to said support via a bond between said DMA and an amino group on said protein;
contacting said protein of interest on said solid support with an ATRP initiator, wherein said ATRP initiator is NETS-Br, and wherein said NHS—Br modifies amino groups on said protein of interest;
coupling repeating subunits of a polymer to said protein of interest via said ATRP initiator, thus generating said protein-polymer conjugate; and
releasing said protein-polymer conjugate from said solid support by exposing said protein-polymer conjugate on said solid support to a pH between about 3 and about 4, such that said protein-polymer conjugate is released from said solid support.

34. The method of claim 33, wherein said protein of interest is an enzyme, a hormone, a cytokine, an antibody, an antigen, an or anti-coagulation protein.

35. The method of claim 33, wherein said protein of interest is an enzyme selected from the group consisting of a protease, an esterase, a dehydrogenase, a hydrolase, and a kinase.

36. The method of claim 33, comprising coupling said protein of interest to said solid support at a pH between 5.5 and 6.5, such that said protein of interest is attached to the solid support via a covalent bond formed between said DMA and said N-terminal amino group of said protein.

37. The method of claim 33, further comprising, prior to exposing the protein-polymer conjugate on said solid support to a pH between about 3 and about 4, contacting the protein-polymer conjugate on said solid support with agarase.

38. The method of claim 33, wherein said protein of interest is an enzyme, and wherein said method further comprises, prior to or simultaneously with contacting said protein of interest with said initiator, said repeating subunits of said polymer, or both, contacting said protein of interest with a substrate for said enzyme.

39. The method of claim 33, wherein said polymer is a zwitterionic polymer.

40. The method of claim 33, wherein said polymer comprises a methacrylate, acrylate, acrylamide, styrenic, or acrylamide-styrenic or a combinations thereof.

41. The method of claim 33, wherein said polymer is pCBMA.

42. The method of claim 33, further comprising washing excess initiator away from said protein of interest on said solid support, prior to coupling said repeating subunits of said polymer to said protein of interest.

43. The method of claim 33, wherein said attaching, contacting, and coupling steps are conducted in an automated system.

44. The method of claim 43, wherein said automated system is a flow through system.

45. The method of claim 33, wherein said method comprises:
washing uncoupled protein of interest away from said solid support prior to adding said initiator,
washing excess initiator away from said protein of interest on said solid support prior to coupling said repeating polymer subunits to said protein of interest, and
washing uncoupled polymer subunits away from said protein-polymer conjugate on said solid support prior to said releasing.
